(19) United States
(12) Patent Application Publication
(10) Pub. No.: US 2006/0161118 A1
(43) Pub. Date: Jul. 20, 2006

(54) Dysfunction resistant catheter system and associated methods

(75) Inventors: Thomas J. Maginot, Crown Point, IN (US); Paul J. Maginot, Fishers, IN (US); James C. Barnitz, Schwenksville, PA (US); Richard M. Derman, Holland, PA (US); Kristopher P. Kundra, Lambertville, NJ (US); Michael J. Rello, Harleysville, PA (US); David M. Strome, Newtown, PA (US)

Correspondence Address: Paul J. Maginot, 10269 Bent Creek Court, Fishers, IN, 46037, US

(73) Assignee: CathLogic, Inc., Crown Point, IN (US)

(21) Appl. No.: 11/375,856

(22) Filed: Mar. 15, 2006

Related U.S. Application Data
(63) Continuation-in-part of application No. 10/857,185, filed on May 28, 2004.
(63) Continuation-in-part of application No. 10/857,621, filed on May 28, 2004.

(51) Int. Cl. A61M 5/00

(52) U.S. Cl. 604/271

(57) ABSTRACT

A catheter system includes a structure defining a proximal orifice, a distal orifice, and a fluid passage extending therebetween. The catheter system further includes an actuator movable in relation to the structure between a first location and a second location. In addition, the catheter system includes a member movable in relation to the structure between (i) a first position in which the member extends through the distal orifice so that a proximal part of the member is positioned within the fluid passage and a distal part of the member is positioned outside of the fluid passage, and (ii) a second position in which the member is entirely located within the fluid passage. The catheter system additionally includes a linkage connected between the actuator and the member. Movement of the actuator from the second location to the first location causes movement of the member from the second position to the first position.

This application is a continuation-in-part of both (i) co-pending application Ser. No. 10/857,621, filed on May 28, 2004 (Patent Application Publication No. US 2005/0059925 A1), and (ii) co-pending application Ser. No. 10/857,185, filed on May 28, 2004 (Patent Application Publication No. US 2005/0096609 A1). The disclosures of each of the above-identified patent applications are hereby totally incorporated by reference in their entirety.

### CROSS REFERENCE

Cross reference is made to the following U.S. Pat. Nos. 5,989,213; 6,156,016; 6,190,371; 6,475,207; 6,585,705; 6,723,084; 6,743,218; 7,008,412, the disclosures of each of the above-identified patents is hereby totally incorporated by reference in their entirety. Cross reference is further made to co-pending application Ser. No. 10/005,277 (Patent Application Publication No. US 2002/0091362 A1, the disclosure of which is hereby totally incorporated by reference in its entirety.

### BACKGROUND

The present disclosure relates generally to catheters, and more particularly to catheter systems for use in a body of a patient that have a higher resistance to dysfunction in relation to existing catheter systems.

Catheters may be used for withdrawal and/or introduction of fluids from a body of a patient. They may be placed at any of various locations in the body such as ducts, cavities, and the vascular system. Such placement depends on the particular use of the catheter. Catheters may have only a single lumen, or alternatively, may have multiple lumens depending on the particular procedure in which it is used. Examples of medical procedures in which a single lumen catheter is placed into the vascular system include (i) chemotherapy or other long-term medicinal infusions, (ii) administration of total parenteral nutrition, (iii) repetitive blood transfusions, and (iv) repetitive blood samplings. Examples of medical procedures in which a multiple lumen catheter is placed into the vascular system includes the performance of hemodialysis or plasmapheresis.

When a catheter is utilized to perform hemodialysis, a physician may place a catheter in the vascular system for a relatively long period of time. In particular, a patient suffering from kidney failure who is involved in a hemodialysis regimen typically requires a dialysis session three days per week for an indefinite period of time whereby extra fluid, chemicals, and wastes are removed from his/her body. A patient who is involved in such a hemodialysis regimen may need a catheter placed in his/her blood vessel for a relatively long period of time in order to provide a ready means for vascular access into his/her bloodstream over such relatively long period of time. This long term placement of the catheter for dialysis purposes may be desirable for a number of reasons.

Firstly, a patient may have experienced progressive loss of other conventional long term vascular access possibilities such as surgically created arteriovenous fistulas. Accordingly, the long term placement of the catheter in the patient's blood vessel may be the best alternative for the patient as he/she proceeds with the hemodialysis regimen.

Additionally, the long term placement of the catheter in the patient's blood vessel may be desirable after initial creation of an arteriovenous fistula in the patient's body. In particular, it is desirable to provide a ready means for vascular access into the patient's bloodstream during a maturation period of the arteriovenous fistula. The maturation period allows the arteriovenous fistula to develop sufficiently so that it will function as a ready means for vascular access into the patient's bloodstream which may be safely punctured multiple times per week for hemodialysis. The length of time of this maturation period is typically on the order of several weeks (e.g. three weeks) to many months (e.g. six months).

Therefore, when performing a hemodialysis procedure, it is common for a physician to use a permanent catheterization technique to place the catheter in a blood vessel of the patient. The permanent catheterization technique typically entails inserting a permanent catheter into a patient's blood vessel using a “tunneled catheter technique.” The tunneled catheter technique includes (i) creating a first opening by making a small incision in a patient's skin with a scalpel directly over the blood vessel to be catheterized, (ii) puncturing the blood vessel at a location directly below the first opening by advancing a needle through the skin incision and subcutaneous tissue and into the blood vessel, (iii) advancing a guidewire through the needle into the blood vessel, (iv) removing the needle over the guidewire, (v) passing one or more tubular vessel dilators over the guidewire to widen the opening defined in the skin and subcutaneous tissue, and further to widen the opening defined in the blood vessel wall to a caliber similar to that of the tubular guide, (vi) advancing the tubular guide over the guidewire and into the blood vessel, (vii) thereafter, creating a second opening in the patient's skin spaced apart at least several centimeters from the first opening, (viii) advancing a tunneling instrument from the second opening to the first opening so as to create a passageway within the subcutaneous tissue under the skin between the first opening and the second opening, (ix) advancing a permanent catheter having a tissue ingrowth member attached to an outer surface thereof into the second opening and through the passageway such that a distal end of the permanent catheter is located adjacent the first opening, (x) inserting the distal end of the permanent catheter through the tubular guide member and into the blood vessel to be catheterized whereby the tissue ingrowth member is positioned in the subcutaneous tissue, (xi) removing the tubular guide member, and (xii) closing the first opening with suture whereby the permanent catheter (a) is no longer exposed through the first opening, (b) extends for at least several centimeters under the patient's skin between the second opening and the location where the permanent catheter enters the blood vessel, and (c) extends out of the second opening so that a proximal end of the permanent catheter is located outside of the patient's body.

The tunneled catheter technique results in the placement of a catheter in a patient's body in a manner which allows the catheter to remain safely in the patient's body for a relatively long period of time. For example, a degree of safety is achieved by separating the following two openings by at least several centimeters: (i) the skin opening through which the catheter enters the patient's body, and (ii) the blood vessel opening through which the catheter enters the patient's vascular system. This safety feature decreases the likelihood that bacteria will migrate up the length of the catheter from the skin opening and cause an infection at the blood vessel opening.

In addition, another degree of safety is achieved by providing a tissue ingrowth member which is attached to and extends around an outer surface of the catheter. As the catheter is left in the patient's body over a period of time, the tissue ingrowth member becomes affixed to the subcutaneous tissue of the patient's body thereby providing a secure attachment of the catheter to the patient's body. Providing a secure attachment between the catheter and the patient's body reduces the likelihood that the catheter will be inadvertently removed or withdrawn from the patient's body. Moreover, since the subcutaneous tissue becomes attached to the tissue ingrowth member, a physical barrier is created between following two openings: (i) the skin opening through which the catheter enters the patient's body, and (ii) the blood vessel opening through which the catheter enters the patient's vascular system. This physical barrier further decreases the likelihood that bacteria will migrate up the length of the catheter from the skin opening and cause an infection at the blood vessel opening.

While the tunneled catheter technique provides the significant advantage of allowing the catheter to remain safely in the patient's body for a relatively long period of time, significant disadvantages of the tunneled catheter technique exist. For example, when a catheter remains in a blood vessel for a long period of time, there is a tendency for blood clots including fibrin (e.g. in the form of a fibrin sheath or sleeve) to attach to and build-up on the outer (and even the inner surfaces adjacent the distal orifices) of the portion of the catheter which is located within the blood vessel. The above described attachment and build-up tends to occlude the various distal orifices defined in the catheter which enable fluid movement into and out of the catheter. For instance, attempts at withdrawing blood through the catheter may be unsuccessful due to blood clots creating a “ball-valve” effect which occlude the various distal orifices of the catheter. Some researchers have found that a fibrin sheath could form as early as twenty-four (24) hours after placement of the catheter in the vascular system. FIG. 1 shows a conventional dialysis catheter 2 placed within the vascular system 4 of a patient having a fibrin sheath 6 formed thereon. Note that after formation, the fibrin sheath 6 covers the entire outer surface of the portion of the catheter that is located in the vascular system 4, and extends from the distal end of the catheter to its entry into the vascular system 4 at venotomy 8. (Note that the lower portions of the catheter 2 and the fibrin sheath 6 are shown in cross section for clarity of understanding.)

When occlusion of the various distal orifices of the catheter occurs due to fibrin sheath formation, a physician has several options for eliminating the occlusion thereby reestablishing access to the vascular system. One option is to remove the occluded catheter and replace it with a new catheter. However, exchanging a catheter which was placed in the patient's body using the tunneled catheter technique is complicated and invasive. Indeed, in order to remove the occluded catheter from the patient's body, the physician must surgically dissect the tissue ingrowth member from the patient's subcutaneous tissue. Recall that the tissue ingrowth member becomes affixed to the subcutaneous tissue over a period of time. Thereafter, the physician would place a new catheter into the patient's body generally using the above described tunneled catheter technique. Thus, this option is undesirable since it requires additional surgery which further traumatizes the patient and increases the cost of medical care.

Another option for eliminating the occlusion of the various distal orifices of the catheter in order to reestablish access to the vascular system involves the performance of a medical procedure in which a blood clot-dissolving medication such as urokinase is infused into the catheter. However, this medication is not always successful in eliminating the occlusion of the various distal orifices of the catheter. In addition, infusion of the medication into the catheter subjects the patient to potential bleeding complications due to the medication entering the vascular system and being circulated systemically. Further, this medication is expensive. Thus, this option has serious drawbacks as well.

An additional option for eliminating the occlusion of the various distal orifices of the catheter in order to reestablish access to the vascular system involves the performance of a medical procedure in which an intravascular snare is introduced into the blood vessel in order to physically strip off any blood clots or fibrin sheath which has attached and built-up on the distal portion of the catheter. However, for catheters placed in veins, this medical procedure requires a venopuncture in the femoral or jugular vein which is invasive and can be uncomfortable for a patient. Furthermore, this option requires the use of (i) an intravascular snare, (ii) a physician experienced in catheter techniques, and (iii) an angiographic suite to provide fluoroscopic imaging. Use of each of items (i), (ii), and (iii) above causes this option to be relatively expensive. Consequently, this option also has significant disadvantages.

What is needed therefore is a catheter system having improved resistance to dysfunction due to occlusion of its various distal orifices. What is also needed is an improved long-term catheter system and associated method of maintaining fluid flow in the catheter system, especially one that has been placed in a patient's body using the tunneled catheter technique.

### SUMMARY

In accordance with one embodiment, there is provided a catheter system that includes a structure defining a proximal orifice, a distal orifice, and a fluid passage extending therebetween. The catheter system further includes an actuator movable in relation to the structure between a first location and a second location. In addition, the catheter system includes a member movable in relation to the structure between (i) a first position in which the member extends through the distal orifice so that a proximal part of the member is positioned within the fluid passage and a distal part of the member is positioned outside of the fluid passage, and (ii) a second position in which the member is entirely located within the fluid passage. The catheter system additionally includes a linkage connected between the actuator and the member. Movement of the actuator from the second location to the first location causes movement of the member from the second position to the first position.

Pursuant to another embodiment, there is provided a catheter system, comprising that includes a structure defining a proximal orifice, a distal orifice, and a fluid passage. The catheter system further includes an actuator movable in relation to the structure. Also, the catheter system includes a member movable in relation to the structure between (i) a first position in which the member is at least partially advanced outside of the structure through the distal orifice so that a distal end of the member is located at a first location outside of the fluid passage, and (ii) a second position in which the distal end of the member is located at a second location which is proximal to the first location. Movement of the actuator causes movement of the member from the second position to the first position.

According to still another embodiment, there is provided a catheter system that includes a structure defining a proximal orifice, a distal orifice, and a fluid passage. The catheter system further includes an actuator movable in relation to the structure. The catheter system also includes a member movable in relation to the structure between (i) a first position in which the member is at least partially advanced outside of the structure through the distal orifice so that a distal end of the member is located outside of the fluid passage, and (ii) a second position in which the distal end of the member is located within the fluid passage. In addition, the catheter system includes a linkage connected between the actuator and the member. Movement of the actuator causes movement of the member from the second position to the first position.

### BRIEF DESCRIPTION OF THE DRAWINGS

FIG. 1 is a fragmentary, partial elevational view, partial cross sectional view, of a prior art dialysis catheter positioned in the vascular system of a patient showing a fibrin sheath formed on the catheter (Note that the lower portions of the catheter and the fibrin sheath are shown in cross section for clarity of understanding);

FIG. 2 is a perspective view of a patient undergoing a dialysis procedure utilizing the catheter system of the present disclosure;

FIG. 3 is a schematic view of a portion of the vascular system of the patient of FIG. 2, showing the right internal jugular vein, the right subclavian vein, the right innominate vein, and the superior vena cava;

FIG. 4 is an enlarged elevational view of the catheter system of FIG. 2;

FIG. 5 is a fragmentary, cross sectional view of a proximal portion of the catheter system of FIG. 2, with the proximal spring of the catheter system shown in its expanded configuration and with the actuator tube shown in its upper position;

FIG. 6 is a view similar to FIG. 5, but showing the proximal spring in its compressed configuration and the actuator tube in its lower position,

FIG. 7 is a view similar to FIG. 6, but showing a fluid line of a dialysis machine coupled to the proximal portion of the catheter system;

FIG. 8 is a view similar to FIG. 5, but showing a cap attached to the proximal portion of the catheter system;

FIG. 9 is a fragmentary, cross sectional view of a distal portion of the catheter system of FIG. 2, with the distal spring of the catheter system shown in its compressed configuration and with the conduit or cage segment shown in its upper position;

FIG. 10 is a view similar to FIG. 9, but showing the distal spring in its expanded configuration and the conduit or cage segment in its lower position;

FIG. 11 is a fragmentary, elevational view of the catheter system of FIG. 2, with a cap shown attached to each of the proximal portions of the catheter system;

FIG. 12 is a fragmentary, cross sectional view of an alternative embodiment of a distal portion of the catheter system of FIG. 2, with the distal spring of the catheter system shown in its compressed configuration;

FIG. 13 is a view similar to FIG. 12, but showing the distal spring in its expanded configuration;

FIG. 14 is an elevational view of the distal spring of FIG. 12;

FIG. 15 is an enlarged view which is similar to FIG. 3, but showing the catheter system of FIG. 2 (i) extending from the right upper chest, (ii) tunneled under the skin within the subcutaneous tissue of the patient for a distance, (iii) entering a venotomy in the right internal jugular vein, and (iv) passing caudally in the right internal jugular vein, the right innominate vein and the superior vena cava;

FIG. 16 is an enlarged view similar to FIG. 15, but depicting the conduit or cage segments of the distal portion of the catheter system in their retracted or upper positions thereby being out of contact with the flow of blood in the patient's vascular system;

FIG. 17 is a view similar to FIG. 16, but depicting the conduit or cage segments of the distal portion in their extended or lower positions thereby being in contact with blood flow in the patient's vascular system such as during performance of a dialysis procedure;

FIG. 18 is an elevational view of an alternative embodiment of the catheter system of FIG. 2, with the distal springs of the distal portion of the catheter system in their compressed or upper positions so as to be out of view of an observer;

FIG. 19 is a fragmentary, cross sectional view of a proximal portion of the catheter system of FIG. 18, with the proximal spring of the catheter system shown in its expanded configuration and with the actuator tube shown in its upper position;

FIG. 19A is a view similar to FIG. 19, but showing a cap attached to the proximal portion of the catheter system;

FIG. 20 is a view similar to FIG. 19, but showing a fluid line of a dialysis machine coupled to the proximal portion of the catheter system;

FIG. 21 is a fragmentary, cross sectional view of a distal portion of the catheter system of FIG. 18, with the distal springs of the catheter system shown in their compressed configuration;

FIG. 22 is a view similar to FIG. 21, but showing the distal springs in their expanded configuration;

FIGS. 23A, 23B and 23C are various perspective views of the actuator tube of the catheter system of FIG. 18;

FIGS. 24A, 24B and 24C are various perspective views of the tube adapter of the catheter system of FIG. 18;

FIGS. 25A, 25B and 25C are various perspective views of the female Luer adapter of the catheter system of FIG. 18;

FIG. 26A is an elevational view of the linkage of the catheter system of FIG. 18 showing its connection between the proximal portion of the catheter system and the distal portion of the catheter system;

FIG. 26B is a partial elevational view (upper part), partial cross sectional view (lower part) showing a lower portion of the linkage of FIG. 26A as well as the distal spring of the catheter system of FIG. 26A (FIG. 26B is also a cross sectional view taken along the line A-A of FIG. 26C);

FIG. 26C is an elevational view of the distal end of the distal spring and bonding washer of FIG. 26A

FIG. 27 is an enlarged fragmentary view of the catheter system of FIG. 18, showing in phantom a portion of the linkage of FIG. 26A;

FIG. 28 is a view similar to FIG. 26A, but showing an alternative embodiment of a linkage that may be used in the catheter system of FIG. 18 instead of the linkage shown in FIG. 26A.

FIG. 29 is a fragmentary, cross sectional view of the tube segment of the distal portion of the catheter system of FIG. 18, with various components of the catheter system removed for clarity of understanding including the distal springs, the bonding washers, and the linkages;

FIG. 29A is a cross sectional view taken along the line 29A-29A of FIG. 29;

FIG. 29B is a cross sectional view taken along the line 29B-29B of FIG. 29;

FIG. 29C is a cross sectional view taken along the line 29C-29C of FIG. 29;

FIG. 29D is a cross sectional view taken along the line 29D-29D of FIG. 29;

FIG. 30 is a fragmentary, cross sectional view of an alternative embodiment of the distal portion of the catheter system of FIG. 18;

FIG. 31 is a fragmentary, cross sectional view of another alternative embodiment of the distal portion of the catheter system of FIG. 18;

FIG. 32 is a fragmentary, cross sectional view of yet another alternative embodiment of the distal portion of the catheter system of FIG. 18; and

FIG. 33 is a view similar to FIG. 29, but showing an alternative embodiment of the tube segment of the distal portion of the catheter system of FIG. 18.

### DESCRIPTION OF THE PREFERRED EMBODIMENTS

While the catheter system described herein is susceptible to various modifications and alternative forms, specific embodiments thereof have been shown by way of example in the drawings and will herein be described in detail. It should be understood, however, that there is no intent to limit the catheter system to the particular forms disclosed, but on the contrary, the intention is to cover all modifications, equivalents, and alternatives falling within the spirit and scope of the invention as defined by the appended claims.

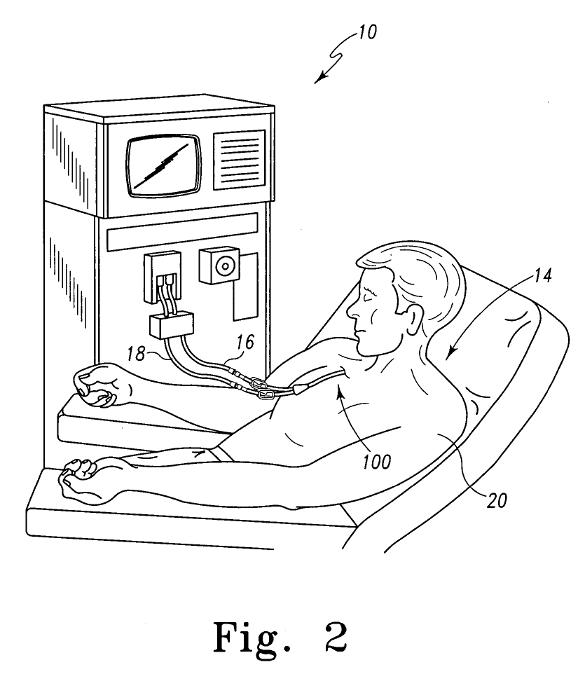

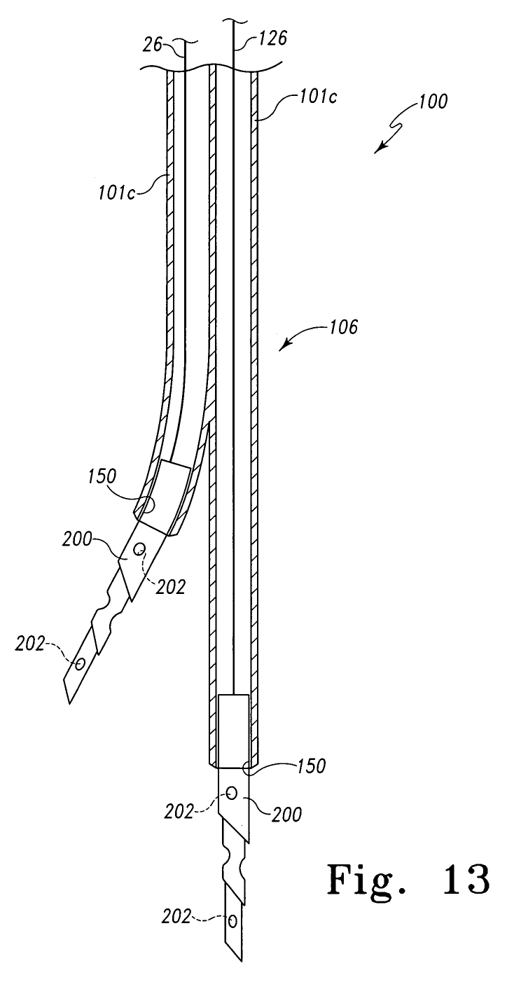

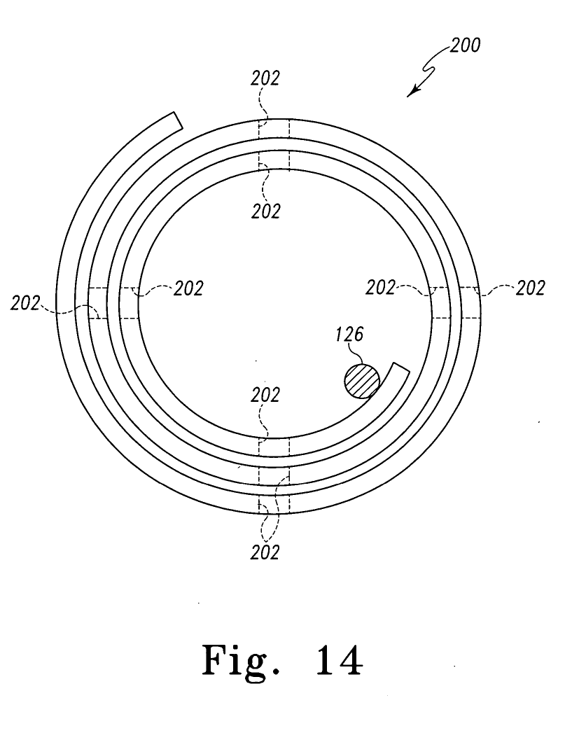

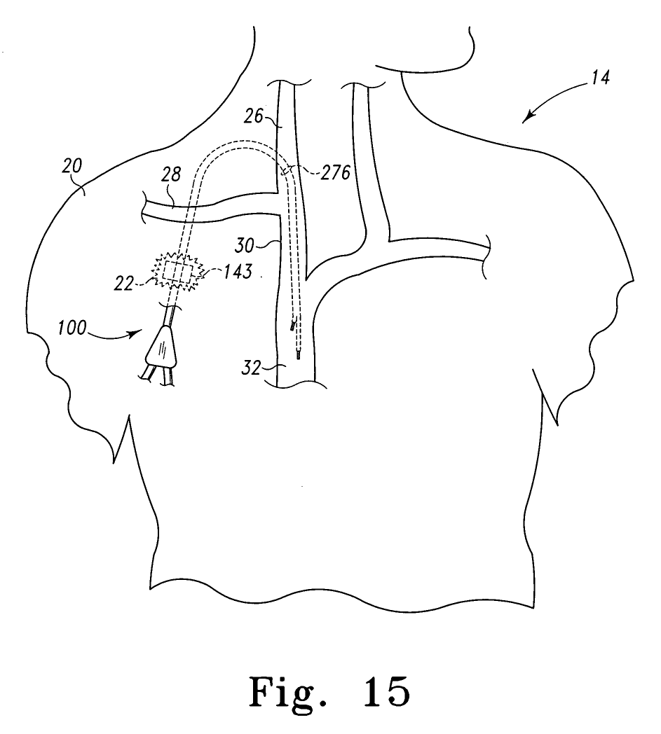

Referring now to FIG. 2, there is shown a hemodialysis machine 10 to which is attached a catheter system 100. The catheter system 100 is inserted in a patient's body 14. The hemodialysis machine 10 includes an inlet line 16 and an outlet line 18 which are each in fluid communication with the catheter system 100. The body 14 includes skin, generally indicated by the reference numeral 20. The body 14 further includes subcutaneous tissue 22 positioned below the skin 20 (see, e.g., FIG. 15).

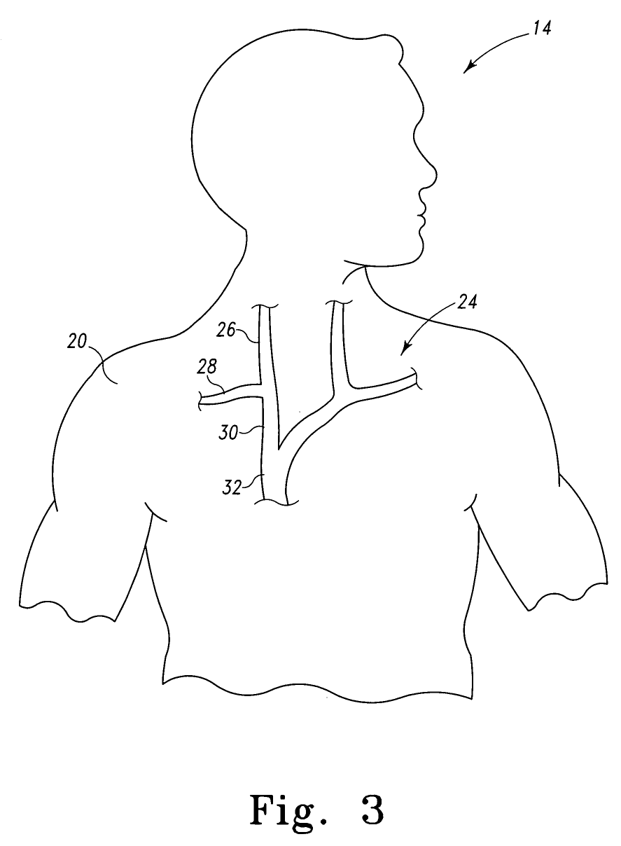

As shown in FIG. 3, the body 14 further includes a vascular system 24. The vascular system 24 includes a right internal jugular vein 26, a right subclavian vein 28, a right innominate vein 30, and a superior vena cava 32. Note that the vascular system 24 is positioned within the body 14 underneath the skin 20. However, the vascular system 24, including the right internal jugular vein 26, the right subclavian vein 28, the right innominate vein 30, and the superior vena cava 32, are depicted in FIGS. 3 and 15-17 with solid lines for clarity of description.

I. Catheter System 100

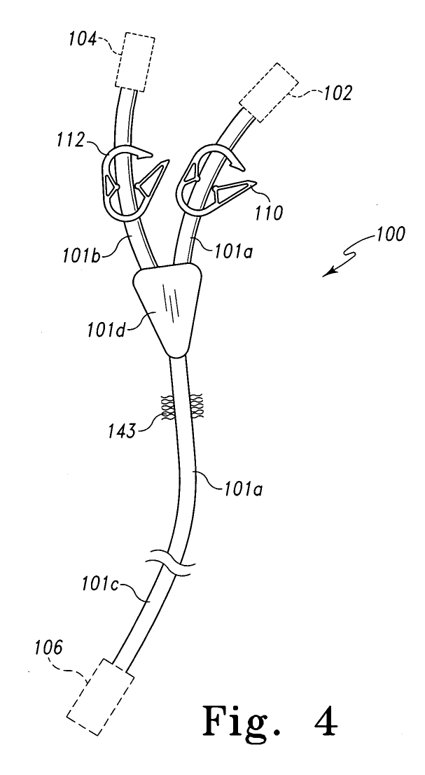

The catheter system 100 is shown in more detail in FIG. 4. In particular, the catheter system 100 includes a first tube segment 101a, a second tube segment 101b, a third tube segment 101c, and a hub 101d. The catheter system 100 further includes a first proximal portion 102, a second proximal portion 104, and a distal portion 106. The first proximal portion 102 is identical in construction and operation to the second proximal portion 104. A tissue ingrowth member 143 is positioned around and secured to an outer surface of the third tube segment 101c of the catheter system 100 as shown in FIG. 4. The tissue ingrowth member 143 is identical to the tissue ingrowth members described in any of U.S. Pat. Nos. 5,989,213; 6,156,016; 6,190,371; 6,475,207; 6,585,705; 6,723,084; 6,743,218; and 7,008,412, as well as, U.S. Patent Application Publication Nos. 2005/0096609 A1 and 2005/00559925 A1. Furthermore, the catheter system 100 includes clamps 110, 112 as shown in FIG. 4, and these clamps are configured and used in the same manner as the clamps described in any of U.S. Pat. Nos. 5,989,213; 6,156,016; 6,190,371; 6,475,207; 6,585,705; 6,723,084; 6,743,218; and 7,008,412, as well as, U.S. Patent Application Publication Nos. 2005/0096609 A1 and 2005/00559925 A1.

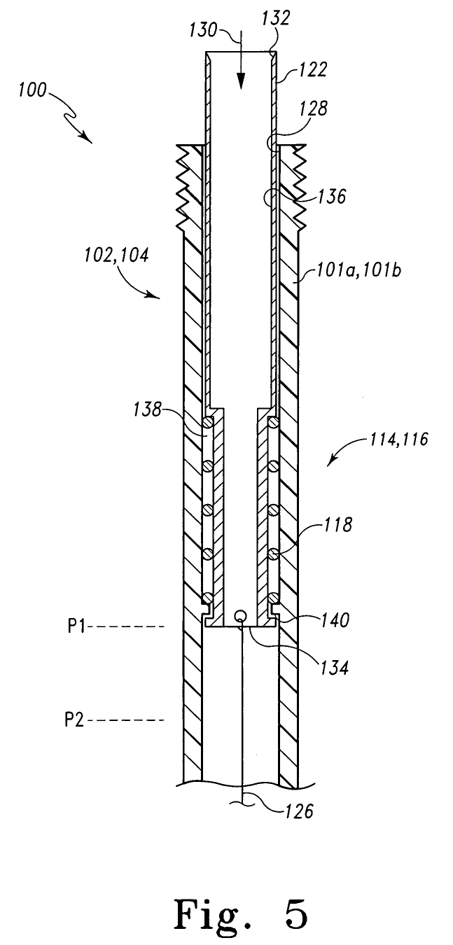

Turning now to FIGS. 5-10, the catheter system 100 further includes a first retractable conduit or cage assembly 114 and a second retractable conduit or cage assembly 116. Note that the first retractable conduit assembly 114 is substantially identical in construction and operation to the second retractable conduit assembly 116. Thus, only the first retractable conduit assembly will be discussed in detail hereinbelow.

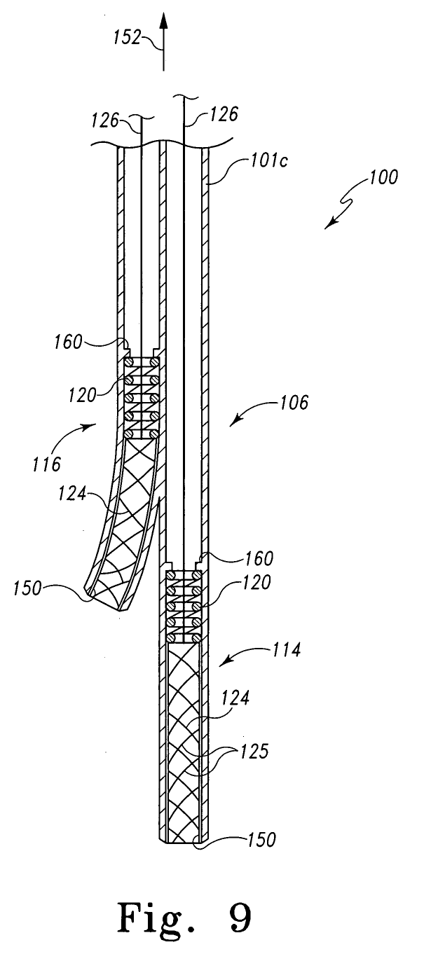

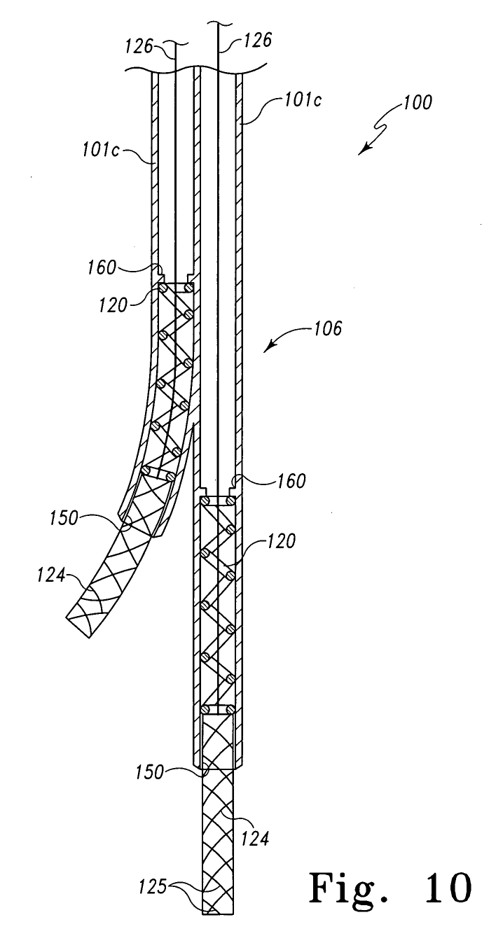

The first retractable conduit assembly 114 includes a proximal spring 118 (see FIGS. 5-8) and a distal spring 120 (see FIGS. 9-10). The springs 118, 120 are preferably made from a metallic material such as stainless steel or titanium or other biocompatible metallic material, but may also be made from other biocompatible materials such as a plastic material. The springs 118, 120 may also be made from Nitinol. The first retractable conduit assembly 114 further includes an actuator tube 122 positioned in the tube segment 101a, 101b as shown in FIGS. 5-8. The first retractable conduit assembly 114 additionally includes a conduit or cage segment 124 secured to the distal spring 120 (see FIGS. 9-10). The conduit assembly 124 is positioned in the tube segment 101c as shown in FIGS. 9-10. The first retractable conduit assembly 114 also includes a cable or wire or linkage 126 that is attached at its proximal end to the actuator tube 122 (see FIGS. 5-8), and further attached at its distal end to the proximal end of the conduit segment 124 (see FIGS. 9-10). The cable 126 may be made of a metallic material such as titanium or stainless steel (or other biocompatible metals) or a plastic material such as polyethylene, polyurethane, or polypropylene (or other biocompatible plastics) or any other biocompatible material such as the material commonly used to make sutures in the medical arts. The cable 126 may also be made from a nylon monofilament material. The cable 126 may be configured as a Bowden cable such as the Bowden cable described in U.S. Patent Application Publication Nos. 2005/0096609 A1 and 2005/00559925 A1.

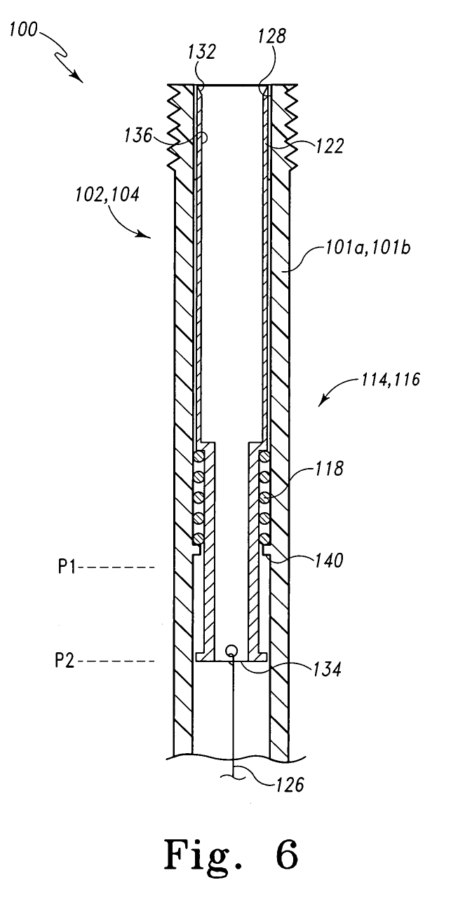

The actuator tube 122 is configured to allow fluid, such as blood, to flow therethrough. To this end, the actuator tube 122 includes a proximal opening 132, a distal opening 134, and a lumen 136 extending therebetween. The proximal spring 118 is positioned around the actuator tube 122 as shown in FIG. 5-8. The actuator tube 122 is movable between an upper position shown in FIG. 5 and a lower position shown in FIG. 6. The actuator tube 122 assumes its upper position absent application of external force thereto. In particular, the proximal spring 118 biases the actuator tube 122 to its upper position as shown in FIG. 5. In this position, a proximal part of the actuator tube 122 is located above or extends out through a proximal orifice 128 of the tube segment 101a, 101b as shown in FIG. 5. Then, upon application of a downwardly directed force against the actuator tube 122 in the direction of arrow 130 in an amount sufficient to overcome the spring bias of the proximal spring 118, the actuator tube is moved from its upper position as shown in FIG. 5 to its lower position as shown in FIG. 6. As a result, the proximal spring 118 is compressed from an expanded configuration as shown in FIG. 5 to a compressed configuration as shown in FIG. 6. This occurs because the actuator tube 122 is configured with a narrowed section that defines a spring space 138 in which the proximal spring 118 is positioned during operation of the catheter system 100. Upon application of force to the actuator tube 122 in the direction 130, the tube 122 is moved downwardly. However, the distal end of the proximal spring 118 is prevented from concurrently being moved downwardly due to a flange 140 that extends inwardly from an interior surface of the tube segment 101a, 101b as shown in FIGS. 5-6. As can be seen in FIGS. 5-6, movement of the actuator tube from its upper position shown in FIG. 5 to its lower position shown in FIG. 6 causes a proximal end of the cable 126 to move from an upper position P1 as shown in FIG. 5 to a lower position P2 as shown in FIG. 6.

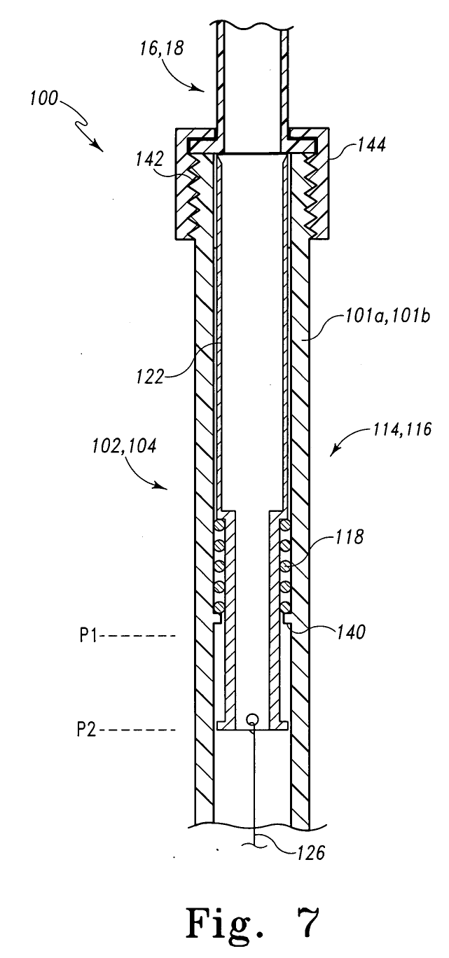

FIG. 7 shows the first proximal portion 102, 104 of the catheter system 100 connected to a fluid line of a dialysis machine such as line 16, 18 of the dialysis machine 10 (see FIG. 2). In particular, a proximal part of the tube segment 101a, 101b has a coupling 142 schematically depicted as a set of external threads, while the distal part of the line 16, 18 has a coupling 144 that is configured to mate in a fluid tight manner to coupling 142. The coupling 144 is schematically depicted as a cap having a set of internal threads. The couplings 142, 144 are preferably configured as Luer lock couplings which are well know in the medical device arts.

When the catheter system 100 is desired to be connected to the line 16, 18 to perform a medical procedure, such as a dialysis procedure, the distal part of the line 16, 18 is urged against the proximal part of the actuator tube segment 122 until the couplings 142, 144 begin to mate with each other. Continued mating of the couplings 142, 144 results in a fluid tight connection between the catheter system 100 and the line 16, 18 as shown in FIG. 7. In this mated condition, the actuator tube 122 is now positioned in its lower position, and the proximal spring 118 is now in its compressed position. Also, the proximal end of the cable 126 is now in its lower position P2 as shown in FIG. 6.

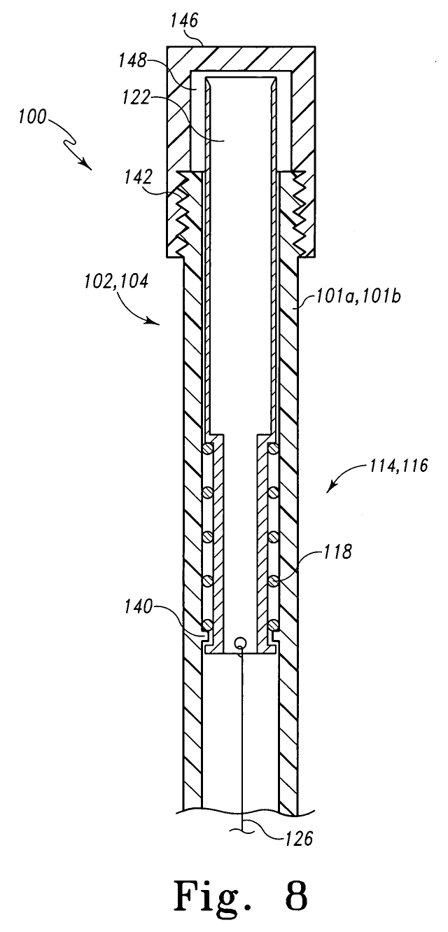

When the catheter system 100 is desired to be disconnected from the line 16, 18 after the medical procedure has been completed, the coupling 144 is manipulated so as to decouple or otherwise separate the couplings 142, 144 from each other. After decoupling, the distal part of the line 16, 18 is moved in a direction away from the proximal part of the tube segment 101a, 101b thereby allowing the actuator tube to be urged by the proximal spring 118 back to its upper position shown in FIG. 5. Thereafter, a cap 146 is secured to the coupling 142 as shown in FIG. 8 in order to seal the catheter system 100 from outside contaminants. The cap includes an elastomeric O-ring (not shown) to effect fluid tight sealing as is well know in the art. Note that cap 146 is configured with an internal space 148 large enough so as to prevent actuation of the actuator tube 122. In other words, when the cap 146 is coupled to the proximal part of the tube segment 101a, 101b as shown in FIG. 8, the proximal spring 118 is allowed to assume its expanded configuration thereby retaining the actuator tube 122 in its upper position. When the actuator tube 122 is retained in its upper position, the proximal end of the cable 126 is retained in its upper position P1. FIG. 11 shows the caps 146 of the proximal portions 102, 104 coupled to their respective proximal parts of the tube segments 101a, 101b. Also, each cap 146 may have an attachment assembly 147 (shown in phantom in FIG. 11) so that the cap 146 will not become misplaced when decoupled from the tube segments 101a, 101b. The attachment assembly 147 includes an arm 147a extending from a gripping surface of the cap and a ring 147b positioned around the respective tube segment 101a, 101b and attached to the arm 147a.

FIGS. 9-10 show the distal portion 106 of the catheter system 100 in more detail. Also, the distal components of the first retractable conduit assembly 114 and the second retractable conduit assembly 116 are shown in more detail in these figures. Since the construction and operation of the retractable conduit assemblies 114, 116 are substantially identical, only the distal components of the first retractable conduit assembly 114 will be discussed in detail.

The first retractable conduit assembly 114 includes the distal spring 120 and the conduit segment 124 which are secured to one another in the manner shown in FIGS. 9-10. In particular, the distal end of the distal spring 120 is secured to the proximal end of the conduit segment 124 so that a fluid flow passage is defined through the center of these two components. Also, the distal end of cable 126 is secured to the proximal end of the conduit segment 124, and can further be secured to the distal end of the distal spring 120. The conduit segment 124 is preferably configured as a stent, such as a stent that is commonly used along with angioplasty procedures to prop open previously occluded coronary arteries. Such stent 124 includes a plurality of intersecting bars 125 that form a wire cage as shown in FIGS. 9-10. The conduit segment or stent 124 is made from a metallic material such as titanium or stainless steel, but can be formed from any biocompatible material, and can even be formed from a plastic material such as polyethylene, polyurethane, or polypropylene. Alternatively, the conduit segment 124 may be configured as a solid cylindrical (i.e. pipe shaped) member (not shown) formed of plastic such as polyethylene, polyurethane, or polypropylene or any other biocompatible material such as the materials used to form conventional dialysis catheters. The conduit segment or stent 124 may also be formed from Nitinol.

As shown in FIGS. 9-10, the conduit assembly 124 is movable between an upper position (FIG. 9) in which the conduit segment is completely contained within the distal part of the tube segment 101c, and a lower position (FIG. 10) in which the conduit segment is partially positioned within the tube segment 101c but is extending out through a distal orifice 150 thereof. In order to position the conduit segment 124 in its upper position as shown in FIG. 9, an upward force is applied to the cable 126 in the direction of arrow 152 in an amount sufficient to overcome the spring bias of distal spring 120 thereby compressing the distal spring 120 into a compressed configuration as shown in FIG. 9, and thus moving the conduit segment 124 to its upper position as shown in FIG. 9. Note that the proximal end of the distal spring 120 is prevented from concurrently being moved upwardly due to a flange 160 that extends inwardly from an interior surface of a distal part of the tube segment 101c as shown in FIG. 9-10.

In order to position the conduit segment 124 in its lower position as shown in FIG. 10, the force being applied to cable 126 in the direction of arrow 152 is removed thereby allowing the distal spring 120 to expand from its compressed configuration to an expanded configuration. Such expansion of the distal spring 120 causes the distal spring to urge the conduit segment 124 to its lower position as shown in FIG. 10.

The catheter system 100 including the first retractable conduit assembly 114 is shown in FIGS. 8, 9, and 11 during a period of non-use such as during a period between dialysis sessions. Then, in order to use the catheter system 100 to perform a medical procedure such as a dialysis procedure, the caps 146 are disconnected from the proximal part of the tube segments 101a, 101b. FIG. 5 shows the proximal portion 102, 104 of the catheter system 100 with its cap 146 removed. Thereafter, the line 16, 18 of the dialysis machine 10 is coupled to the proximal portion 102, 104 of the catheter system 100 thereby causing the proximal components of the first retractable conduit assembly to assume their positions shown in FIG. 7. (FIG. 6 shows the position of the proximal components of the first retractable conduit assembly 114 when the catheter system 100 is coupled to the lines 16, 18 of the dialysis machine, but with the components of the lines 16, 18 removed for clarity of viewing.) Coupling of the line 16, 18 of the dialysis machine 10 to the catheter system 100 also causes the distal components of the first retractable conduit assembly 114 to move from their positions shown in FIG. 9 to their positions shown in FIG. 10. It should be appreciated that the catheter system 100 including the first retractable conduit assembly 114 is shown in FIGS. 7 and 10 during a period of use such as during performance of a dialysis procedure.

It should further be appreciated that the springs 118 and 120 are configured such that spring 118 is the stronger one. In particular, during periods of non-use of the catheter system 100, the bias of the proximal spring 118 overcomes the bias of the distal spring 120 so as to position the actuator tube 122 and the conduit segment 124 in their upper positions as shown in FIGS. 5 and 9. However, when the influence of the spring 118 is removed from the spring 120 (such as when the catheter system 100 is coupled to lines 16, 18), the spring 120 is configured to bias the conduit segment 124 into its lower position as shown in FIG. 10.

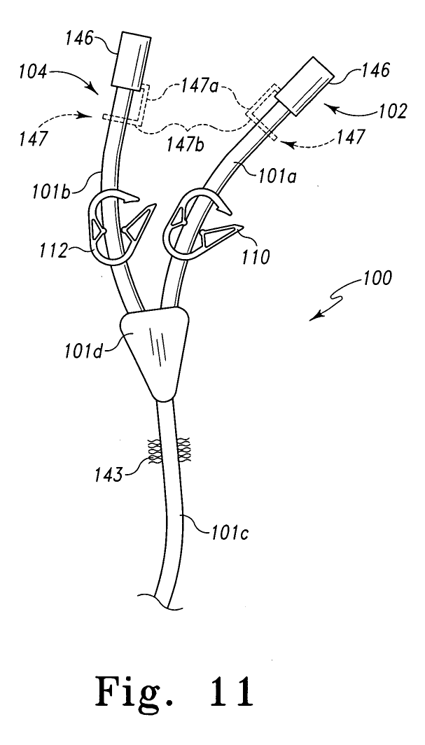

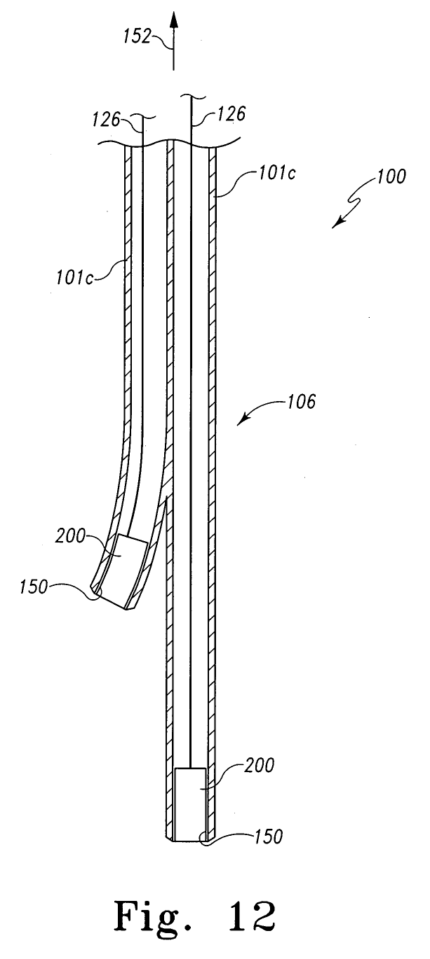

FIGS. 12-14 show an alternative arrangement of the distal portion 106 of the catheter system 100. In particular, instead of the distal portion 106 shown in FIGS. 9-10, the distal portion 106 could be configured to possess a single component that serves both the function of a spring and a conduit or cage. That component is a volute or coil spring 200. The spring 200 is preferably made from a metallic material such as titanium or stainless steel or other biocompatible material. Alternatively, the spring 200 may be formed from a plastic material such a polyethylene, polyurethane, polypropylene or other biocompatible plastic material. The spring 200 may also be made from Nitinol. The spring 200 is secured to the distal part of the tube segment 101c as shown in FIGS. 12-13. The spring 200 possesses a number of fluid openings 202 that extend through the walls of the spring 200. The distal end of cable 126 is secured to the distal end of the spring 200 as shown in FIG. 14.

As shown in FIGS. 12 and 13, the spring 200 is movable between a compressed configuration (FIG. 12) in which the spring 200 is completely contained within the distal part of the tube segment 101c, and an expanded configuration in which the spring 200 is partially positioned within the tube segment 101c but extends out through a distal orifice 150 thereof. In order to position the spring 200 in its compressed configuration as shown in FIG. 12, an upward force is applied to the cable 126 in the direction of arrow 152 in an amount sufficient to overcome the spring bias of spring 200 thereby compressing the spring and moving it completely within the distal part of the tube segment 101c as shown in FIG. 12.

In order to move the spring 200 to its expanded configuration as shown in FIG. 13, the force being applied to cable 126 in the direction of arrow 152 is removed thereby allowing the spring 200 to expand from its compressed configuration to its expanded configuration as shown in FIG. 13. Such expansion of the spring 200 causes the spring 200 to expand and thereby extend out of the distal orifice 150 of the tube segment 101c as shown in FIG. 13.

Note that in this alternative embodiment of the distal portion 106 of the catheter system 100, coupling of the line 16, 18 of the dialysis machine 10 to the catheter system 100 causes the components of the first retractable conduit assembly 114 to move from their positions shown in FIGS. 5 and 12 to their positions shown in FIGS. 7 and 13. Thus, the catheter system 100 including the first retractable conduit assembly 114 is shown in FIGS. 7 and 13 during a period of use such as during the performance of a dialysis procedure.

Also note that in this alternative embodiment, the springs 118 and 200 are configured such that spring 118 is the stronger one. In particular, during periods of non-use of the catheter system 100, the bias of the proximal spring 118 overcomes the bias of the distal spring 200 so as to position the actuator tube 122 and the spring 200 in their upper positions as shown in FIGS. 5 and 12. However, when the influence of the spring 118 is removed from the spring 200 (such as when the catheter system 100 is coupled to lines 16, 18), the spring 200 is configured to expand into its lower or expanded position as shown in FIG. 13 thereby extending out through the distal orifice 150 of the tube segment 101c.

I(a). Placement of the Catheter System 100 Within the Body

The catheter system 100 is placed within the body 14 using the tunneled catheter technique. In particular, a first opening is created by making a small incision in the skin 20 with a scalpel directly over the right internal jugular vein 26. Thereafter, the right internal jugular vein 26 is punctured to create a venotomy 276 (see FIGS. 15-17) at a location directly below the first opening by advancing a needle through the skin incision and the subcutaneous tissue 22 and into the right internal jugular vein 26. Thereafter, a guidewire is advanced through the needle into the right internal jugular vein 26 through the venotomy 276. The needle is then removed over the guidewire. One or more tubular vessel dilators is passed over the guidewire to widen the opening defined in the skin 20 and subcutaneous tissue 22, and further to widen the venotomy 276 defined in the wall of the right internal jugular vein 26 to a caliber similar to that of a tubular guide. Thereafter, the tubular guide is advanced over the guidewire and into the right internal jugular vein 26. Then, a second opening is created in the skin 20 which is spaced apart at least several centimeters from the first opening. A tunneling instrument is advanced from the second opening to the first opening so as to create a passageway within the subcutaneous tissue 22 under the skin 20 between the first opening and the second opening. The catheter system 100 is then advanced into the second opening and through the passageway such that the distal end of the tube segment 101c of the catheter system 100 is located adjacent to the first opening. Note that during the above-described advancement of the catheter system 100, the first and second retractable conduit assemblies 114, 116 are each positioned in their retracted position (i.e. in their positions as shown in FIGS. 8 and 9; and FIGS. 8 and 12).

The distal end of the catheter system 100 is then inserted through the tubular guide member and into the right internal jugular vein 26 so that the tissue ingrowth member 143 is positioned in the subcutaneous tissue 22 (see FIG. 16). Thereafter, the tubular guide member is removed. The first opening is then closed with suture whereby the catheter system 100: (a) is no longer exposed through the first opening, (b) extends for at least several centimeters under the skin 20 between the second opening and the venotomy 276, and (c) extends out of the second opening so that the proximal end of the catheter system 100 is located outside of the body 14 as shown in FIG. 16.

Note that after the catheter system 100 is placed in the vascular system 24 as described above, the catheter system 100 is positioned in the right internal jugular vein 26, the right innominate vein 30, and the superior vena cava 32 as shown in FIG. 16. Moreover, note that as the tissue ingrowth member 143 remains in contact with the subcutaneous tissue 22 over a period of time, the subcutaneous tissue 22 becomes affixed to the tissue ingrowth member 143 thereby securing the catheter system 100 to the body 14. As discussed above, affixation of the tissue ingrowth member 143 to the subcutaneous tissue 22 in the above described manner helps prevent bacterial migration up the catheter system 100 from the second opening to the venotomy 276 thereby preventing serious infection.

1(b). Performance of Dialysis Sessions with the Catheter System 100

Once the catheter system 100 is placed in the body 14 as described above, the catheter system is positioned as shown in FIG. 16. In this position, the first and second retractable conduit assemblies 114, 116 are each positioned in its retracted position (i.e. in their positions as shown in FIGS. 8 and 9; and FIGS. 8 and 12). When a patient desires to be dialyzed (i.e. engage in a dialysis session), the tube segments 101a, 101b are respectively connected to the inlet line 16 and the outlet line 18 of the hemodialysis machine 10 as shown in FIG. 2 (see also FIG. 7).

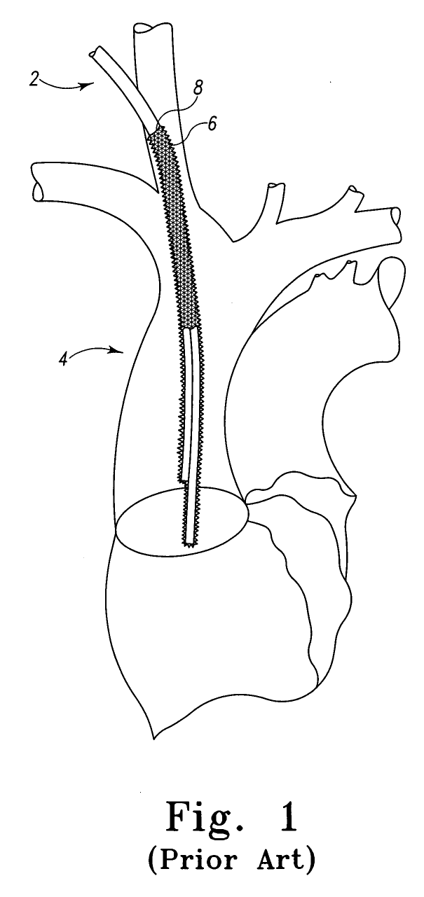

As discussed in detail above, connecting the inlet and outlet lines 16, 18 to the tube segments 101a, 101b as shown in FIG. 7 (see also FIG. 2) causes the first and second retractable conduit assemblies 114, 116 to automatically move from their retracted position to their extended position as shown in FIG. 17. (See e.g., also, extended position as shown in FIGS. 7 and 10; and FIGS. 7 and 13). Moving the first and second retractable conduit assemblies 114, 116 to their extended position causes the conduit or cage segments 124 of the assemblies 114, 116 to be exposed to the blood flow within the superior vena cava 32. (See FIGS. 15, 17; and FIGS. 10 and 13). With the first and second retractable conduit assemblies 114, 116 positioned in the extended position, a dialysis procedure is then performed on the patient's body 14 in a well known manner. Note that when the conduit or cage segments 124 extend out of the distal end of the tube segments 101c in the manner described above, the conduit or cage segments poke through and traverse any fibrin sheath that may have formed on the outer walls of the tube segment 101c. (See, e.g., FIG. 1 which shows a fibrin sheath formed on the outer walls of a conventional dialysis catheter implanted in a patient's vascular system.)

Upon completion of the dialysis procedure, the tube segments 101a, 101b are respectively disconnected from the inlet line 16 and the outlet line 18 of the hemodialysis machine 10. As discussed in detail above, disconnecting the inlet and outlet lines 16, 18 from the tube segments 101a, 101b causes the first and second retractable conduit assemblies 114, 116 to automatically move from their extended positions to their retracted positions (i.e. positions as shown in FIGS. 8 and 9; and FIGS. 8 and 12).

After the lines 16, 18 are disconnected from the catheter system 100, the proximal ends of the tube segments 101a, 101b are then each covered with caps 146 as described above, and the patient is able to carry on about his/her business. Thereafter, when a patient desires to be dialyzed again, the above procedure is repeated.

With the catheter system 100, it should be appreciated that the length of time which the actual working distal end portions of the catheter system are exposed to the blood flow in the superior vena cava 32 is substantially reduced relative to the length of time which the actual working distal end of conventional hemodialysis catheters are exposed. This reduction in blood flow exposure time substantially reduces the likelihood that the distal end of the catheter system 100 will become partially or totally occluded due to attachment or build-up of blood clots, such as fibrin, on the outer and inner surfaces of the distal end of the catheter system 100.

In order to further reduce the likelihood that the distal end portions of the catheter system 100 will become partially or totally occluded due to blood clot attachment or build-up, a quantity of blood clot dissolving liquid may be advanced into the catheter system 100 after a dialysis session is completed in order to flush both fluid flow paths of the catheter system 100 and create a pool in which the distal components of the catheter systems (e.g. the conduit or cage segments 124, the springs 120; and the springs 200) are be bathed. In particular, after the inlet line 16 and the outlet line 18 are disconnected from the proximal end of the catheter system 100 following completion of dialysis session, a quantity of blood clot dissolving liquid is advanced into each of the fluid passages of the dialysis catheter 100 so as to flush both lumens of the catheter system (i.e. venous and arterial lumens). One type of blood clot dissolving liquid which may be used with the present catheter system is urokinase.

After the blood clot dissolving liquid is advanced into the catheter system 100 in the above-described manner, then the proximal end of the catheter system 100 is sealed by connecting the caps 146 to the tube segments 101a, 101b as described above, and subsequently the patient is able to carry on about his/her business. The above flushing procedure may be repeated after each dialysis session is completed.

While advancement of the blood clot dissolving liquid (such as urokinase) into the lumens of the catheter system 100 after a dialysis session has been completed has many advantages, some advantages may also be achieved by advancement of an alternative solution into the catheter system 100 after completion of a dialysis session. For example, instead of advancing blood clot dissolving liquid (such as urokinase) into the catheter system 100 after a dialysis session, a heparin lock flush solution may be advanced into the catheter system 100 after a dialysis session has been completed in order to flush the fluid flow paths of the catheter system 100 and create a pool in which the distal components of the catheter system may be bathed.

It should be noted that the catheter system 100 may further include a distal valve (not shown) positioned adjacent to each distal orifice 150 of the catheter system. Each such distal valve would help maintain the flushing solution (e.g. urokinase or heparin) within the lumens of the catheter system 100 during idle periods when the retractable conduit assemblies 114, 116 are in their retracted positions. Each such distal valve would also help prevent blood which is flowing in the superior vena cava from advancing into the lumens of the catheter system 100 during idle periods in the patient's body when the catheter system is not being used to carry out a dialysis procedure.

It should further be understood that such distal valves would help prevent blood from escaping through the catheter system 100 during idle periods (i.e. after completion of a dialysis session and before commencement of a subsequent dialysis session).

II. Another Alternative Embodiment of Catheter System 100

FIGS. 18-32 show another alternative embodiment of the catheter system 100 of FIG. 4. In this embodiment, the first proximal portion 102, the second proximal portion 104, and the distal portion 106 are constructed as shown in FIGS. 18-32 instead of as shown in FIGS. 5-14. Like the embodiments of FIGS. 5-14, the first proximal portion 102 of the embodiment of FIGS. 18-32 is identical in construction and operation to the second proximal portion 104 of the embodiment of FIGS. 18-32. Also, while the hub 101d of the embodiment of FIGS. 18-32 possesses a slightly different external configuration in comparison to the hub 101d of the embodiments of FIGS. 5-14, its function and internal configuration is essentially the same. The external configuration of the hub 101d of this embodiment is shown in FIG. 18.

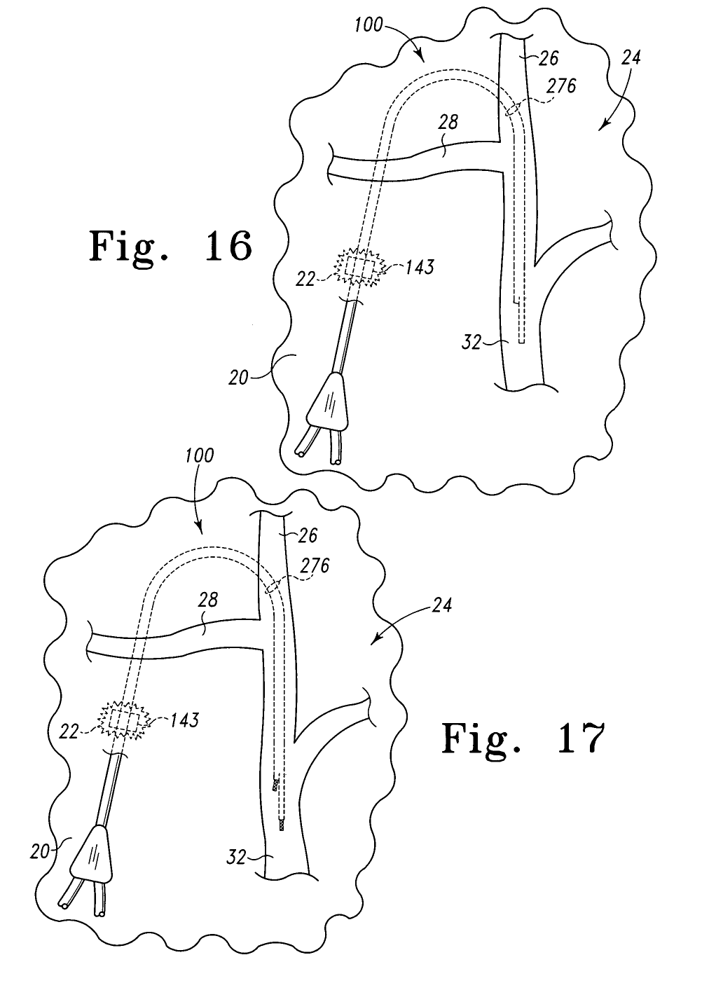

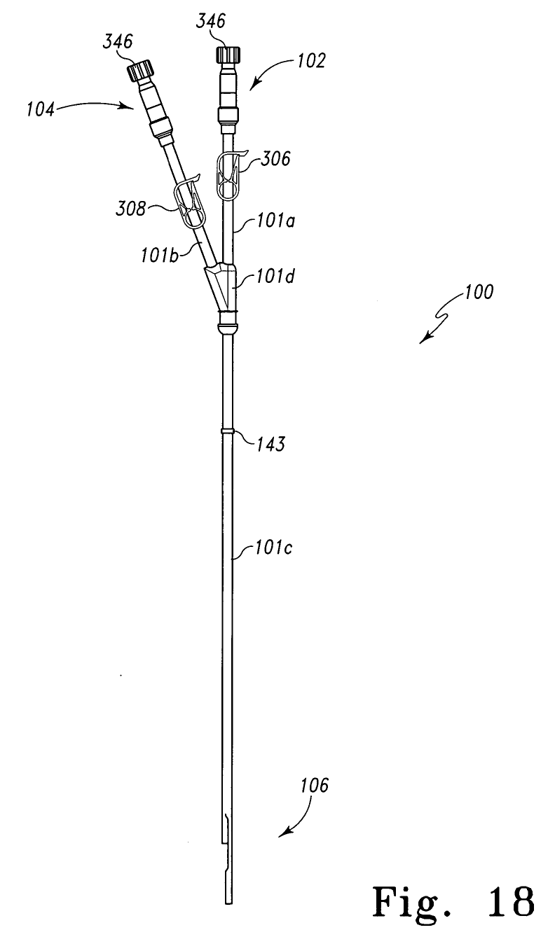

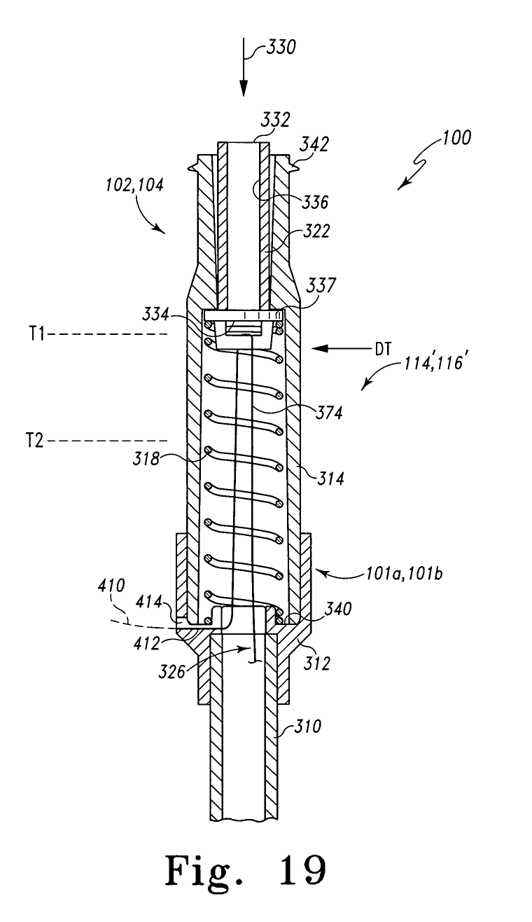

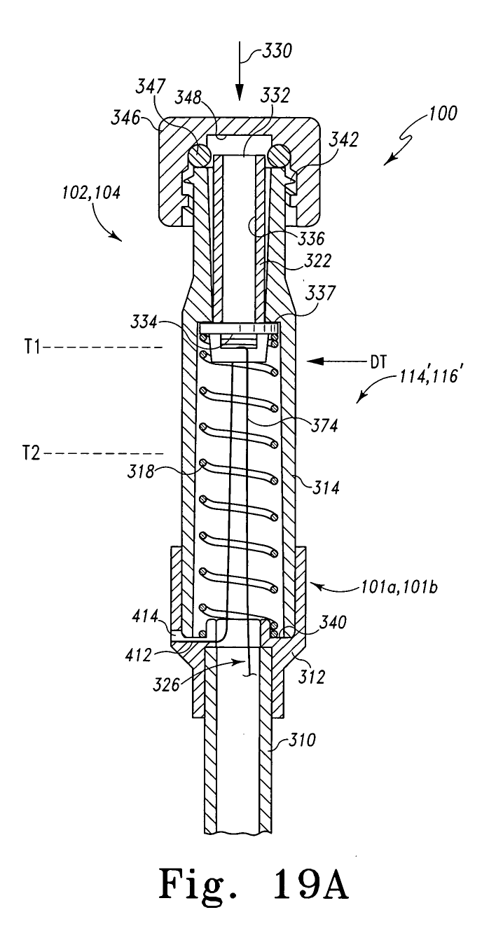

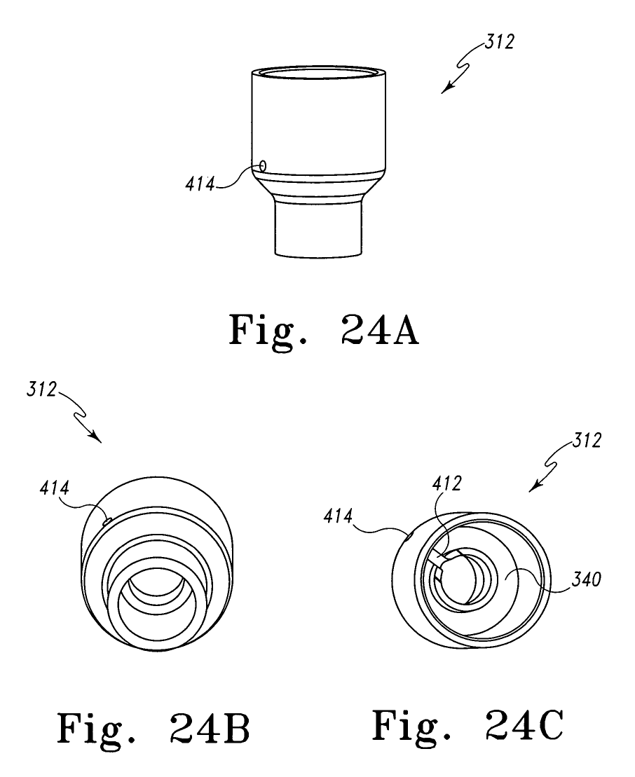

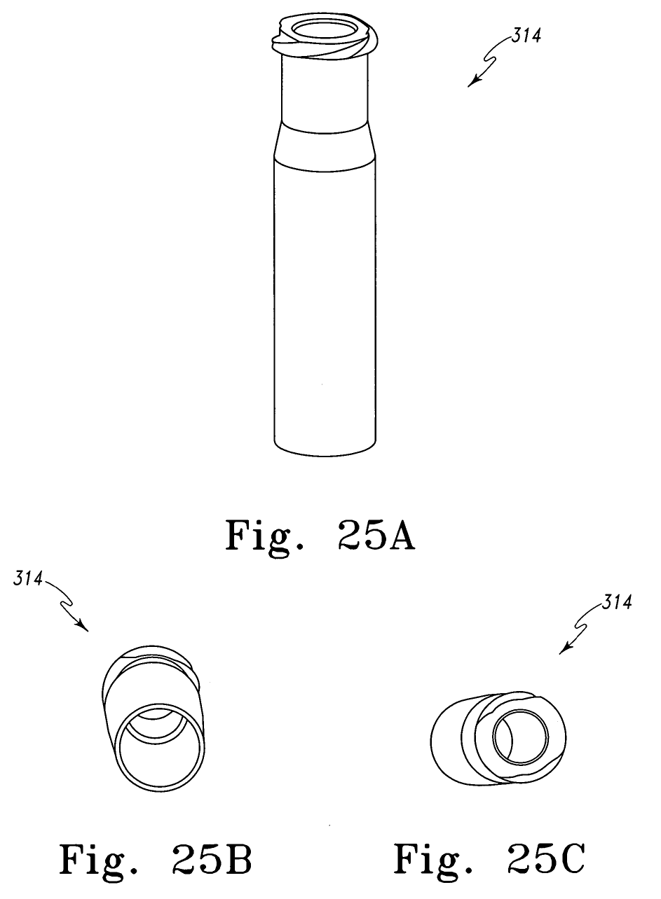

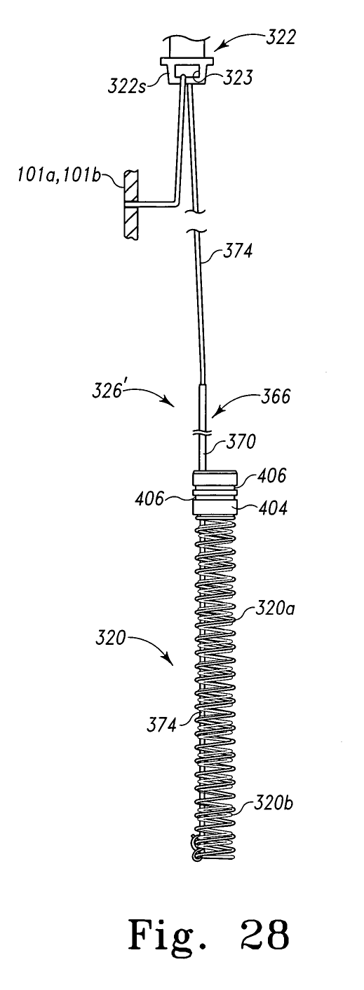

With reference to FIG. 19, the tube segment 101a, 101b includes a tube component 310, a tube adapter 312, and a female Luer adapter 314. The proximal end portion of the tube component 310 is attached to the distal end portion of the tube adapter 312, while the proximal end portion of the tube adapter 312 is attached to the distal end portion of the female Luer adapter 314. When assembled, the tube component 310, the tube adapter 312, and the female Luer adapter 314 defines a conduit through which fluid such as blood may flow. The tube adapter 312 is shown in more detail in FIGS. 24A-24C, while the female Luer adapter 314 is shown in more detail in FIGS. 25A-25C. Clamps 306, 308 are positioned on the tube component 310 of the tube segment 101a, 101b as shown in FIG. 27.

The catheter system 100 of FIGS. 18-32 further includes a first retractable conduit or cage assembly 114′ and a second retractable conduit or cage assembly 116′. Note that the first retractable conduit assembly 114′ is substantially identical in construction and operation to the second retractable conduit assembly 116′. Thus, only the first retractable conduit assembly 114′ will be discussed in detail hereinbelow.

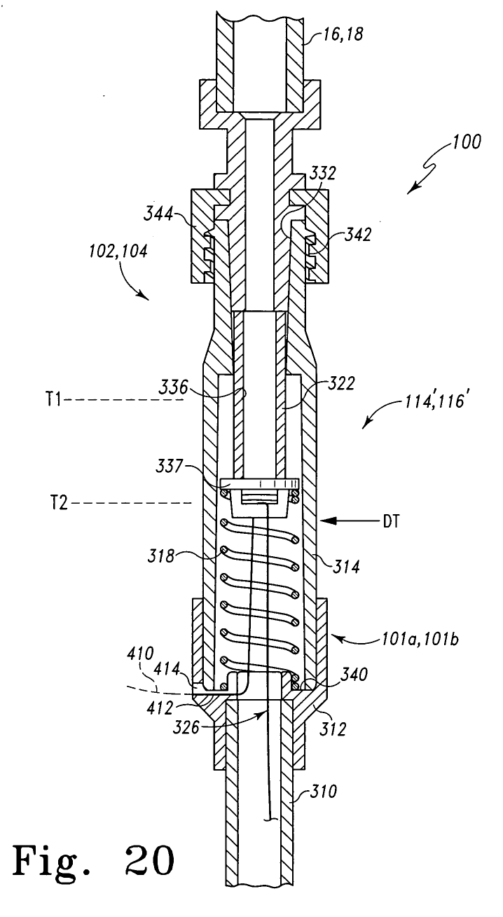

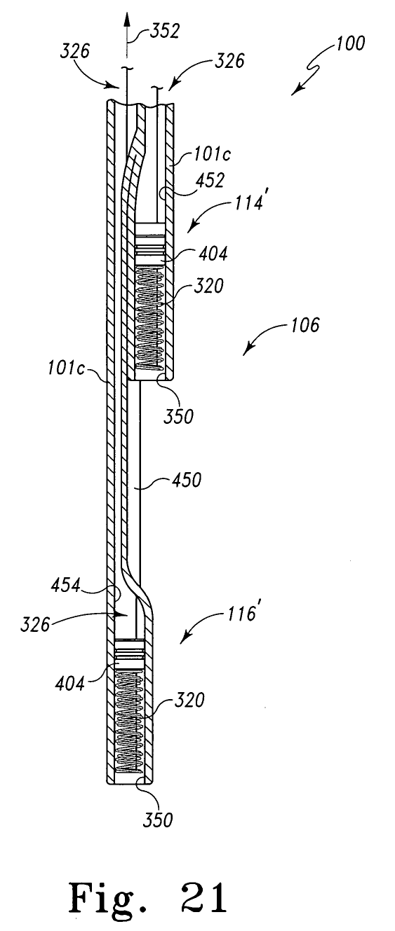

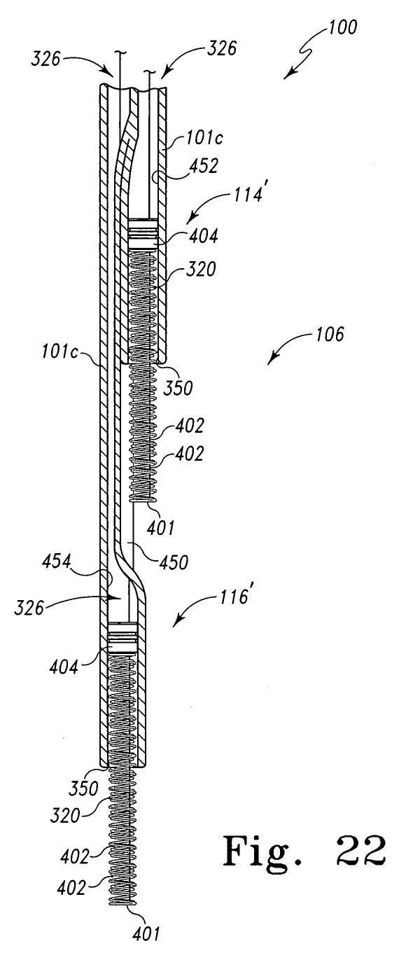

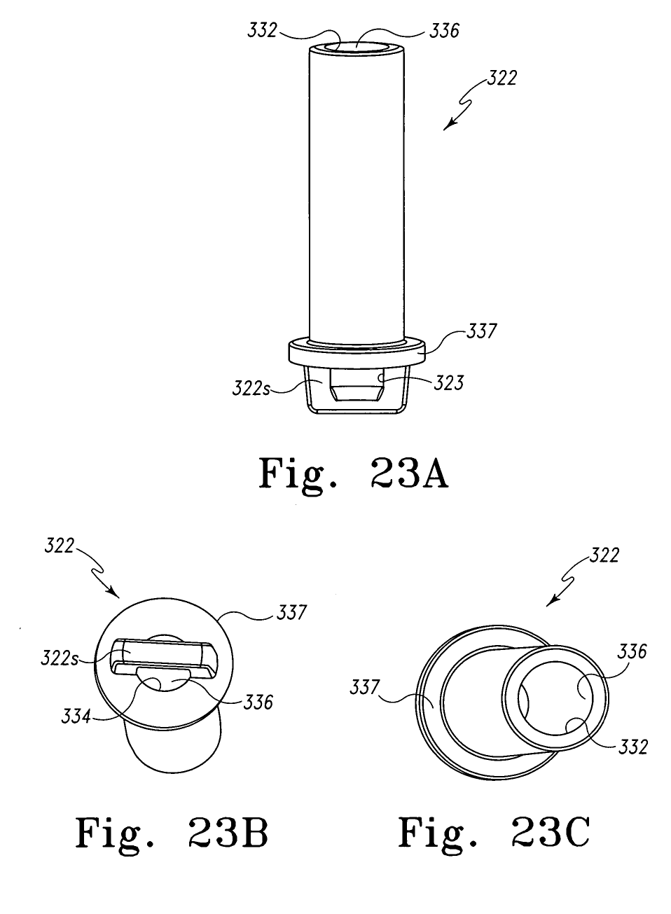

The first retractable conduit assembly 114′ includes a proximal spring 318 (see FIGS. 19-20) and a distal spring 320 (see FIGS. 21-22). The springs 318, 320 are preferably made from a metallic material such as titanium or stainless steel, or other biocompatible metallic material, but may also be made from other biocompatible materials such as a plastic material. The springs 318, 320 may also be made from Nitinol. The first retractable conduit assembly 114′ further includes an actuator tube 322 positioned in the tube segment 101a, 101b as shown in FIGS. 19-20. The actuator tube 322 defines a lumen 336 as is shown in FIGS. 23A-23C. The actuator tube 322 includes a support arm 322s that defines an opening 323. Similar to the embodiment of FIGS. 12-14, the distal spring 320 of the first retractable conduit assembly 114′ (see FIGS. 21-22) is configured as a conduit or cage segment which is positioned in the tube segment 101c as shown in FIGS. 21-22.

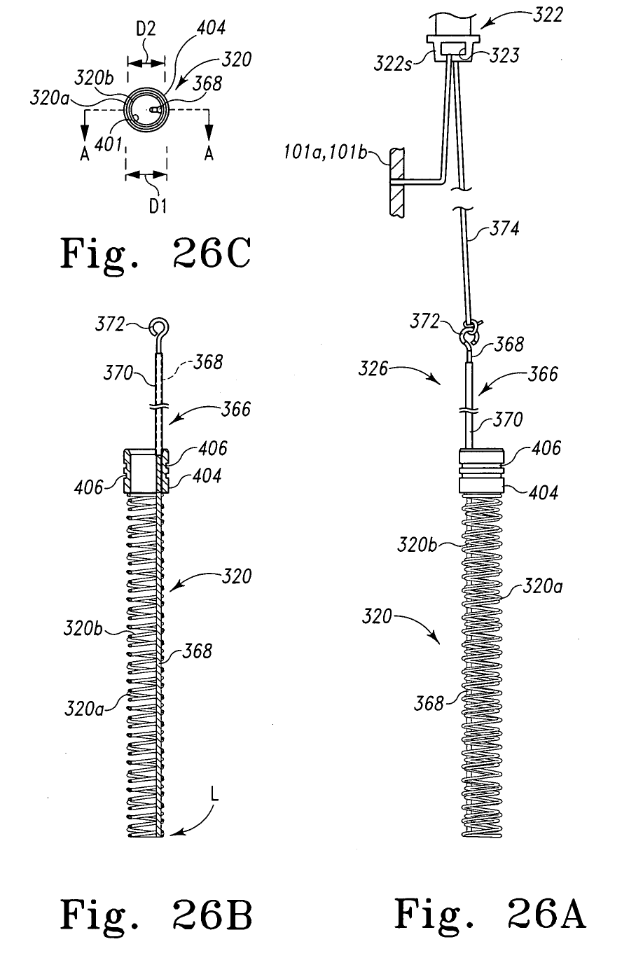

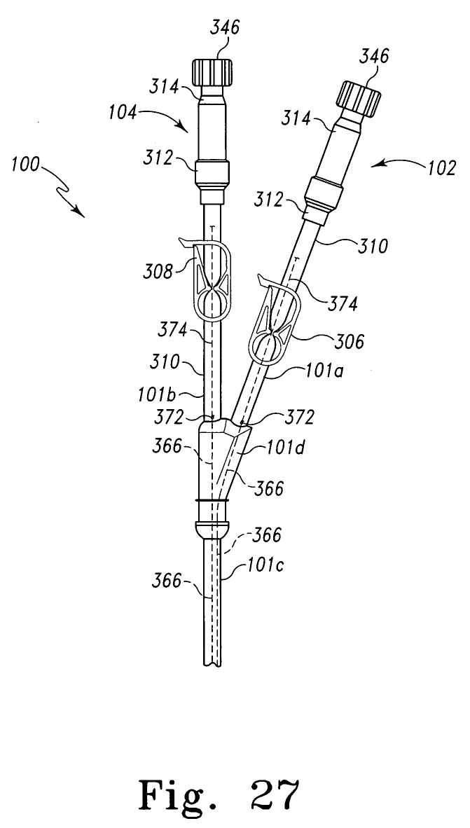

As shown in FIGS. 26A-26C, the spring 320 includes an outer spring component 320a and an inner spring component 320b that are secured to one another, for example, by welding. The outer spring component 320a is wound in a left hand manner and defines a first diameter D1, while the inner spring component 320b is wound in a right hand manner and defines a diameter D2 that is less than D1. (See FIG. 26C.)

The first retractable conduit assembly 114′ also includes a linkage 326 that is attached at its distal end to the distal end of the distal spring 320 at a location L (see FIG. 26C). The linkage 326 is also attached at its proximal end to an internal sidewall of the tube segment 101a, 101b after it loops around the support arm 322s and passes through the opening 323 as shown in FIGS. 19-20. The proximal end of the linkage 326 is secured to the internal sidewall of the tube segment 101a, 101b by threading the proximal end portion through a channel 412 and an aperture 414 defined by the tube adapter 312 and thereafter advancing a cement or glue into the channel and aperture. Excess proximal portions of the linkage may be snipped off so that the proximal end of the linkage 326 is flush with the outer surface of the tube adapter 312 as shown in FIG. 19A.

The linkage 326 is shown in more detail in FIGS. 26A-26C. In particular, the linkage includes a Bowden cable assembly 366 that includes an inner wire or line 368 and an outer sheath 370. The inner wire 368 is at least partially positioned within the outer sheath 370, and the wire 368 and sheath 370 are movable in relation to each other. The inner wire 368 and the outer sheath 370 may be made from a metallic material such as titanium or stainless steel (or other biocompatible metals) or a plastic material such as polyethylene, polyurethane, or polypropylene (or other biocompatible plastics) or any other biocompatible material such as the material commonly used to make sutures in the medical arts. The inner wire 368 may also be made from a nylon monofilament material. The inner wire 368 and the outer sheath 370 need not be made from the same material as each other. Indeed, the inner wire may be made from a first material, and the outer sheath may be made from a second material with is different from the first material. For example, the inner wire may be made from a nylon monofilament material, while the outer sheath may be made from a stainless steel material.

The inner wire 368 of the Bowden cable assembly includes an eyelet 372 formed in a proximal end portion thereof as shown in FIGS. 26A-26C. The linkage 326 further includes a line 374 secured at its distal end portion to the eyelet 372 as shown in FIG. 26A, for example, by way of a tied knot. The line 374 is further secured at its proximal end portion to the inner sidewall of the tube segment 101a, 101b after it loops around the support arm 322s and passes through the opening 323 as shown in FIG. 26A. (See also FIGS. 19-20.) The line 374 is made from a biocompatible material such as the material commonly used in the manufacture of sutures in the medical arts. The line 374 may made from any suitable material such as polypropylene, polyethylene, or polyurethane. Alternatively, the line 374 may be made from a metallic material such as titanium or stainless steel. The line 374 may also be made from a nylon monofilament material.

An alternative linkage 326′ that may be used in the catheter system 100 is shown in FIG. 28. The linkage 326′ shown in FIG. 28 is substantially the same as the linkage shown in FIG. 26A with the exception that the inner wire 368 of the Bowden cable is eliminated and the line 374 extends entirely through the outer sheath 370 and is secured to the distal end of the spring 320, for example, by way of a tied knot. In this alternative embodiment of the linkage 326′, the outer sheath 370 is made of stainless steel, while the line 374 is made of a nylon monofilament material.

In the assembled state of the catheter system 100 of FIGS. 18-32, the line 374 is located in the tube segment 101a, 101b so that closure of the respective clamp 306, 308 pinches the line 374 therein upon closure of the clamp. (See FIG. 27.) Also, in the assembly state, the Bowden assembly 366 is positioned so as to be located well distal to the clamps 306, 308. (See also FIG. 27.) Arranging the Bowden assembly 366 within the catheter system 100 so that it will not be clamped by the clamps 306, 308 ensures that operation of the clamps does not crush or otherwise damage the linkage 326, 326′.

Referring again to FIGS. 19-20, the actuator tube 322 is configured to allow fluid, such as blood, to flow therethrough. To this end, the actuator tube 322 includes a proximal orifice 332, a distal orifice 334, and a lumen 336 extending therebetween. The proximal spring 318 is positioned around the actuator tube 322 as shown in FIG. 19-20. The actuator tube 322 is movable between an upper position shown in FIG. 19 and a lower position shown in FIG. 20. The actuator tube 322 assumes its upper position absent application of external force thereto. In particular, the proximal spring 318 biases the actuator tube 322 to its upper position as shown in FIG. 19. In this position, a proximal part of the actuator tube 322 is located above or extends out through a proximal orifice 332 of the tube segment 101a, 101b as shown in FIG. 19. Then, upon application of a downwardly directed force against the actuator tube 322 in the direction of arrow 330 in an amount sufficient to overcome the spring bias of the proximal spring 318, the actuator tube is moved from its upper position as shown in FIG. 19 to its lower position as shown in FIG. 20. As a result, the proximal spring 318 is compressed from an expanded configuration as shown in FIG. 19 to a compressed configuration as shown in FIG. 20. This occurs because the actuator tube 322 possesses an annular shoulder 337 that contacts the proximal spring 318 upon application of force to the actuator tube 322 in the direction 330. Note that the distal end of the proximal spring 318 is prevented from concurrently being moved downwardly due to interaction of the distal end of the proximal spring 318 and a ledge 340 defined by the tube adapter 312 of the tube segment 101a, 101b as shown in FIGS. 19-20. As can be seen in FIGS. 19-20, movement of the actuator tube 322 from its upper position shown in FIG. 19 to its lower position shown in FIG. 20 causes the location at which the proximal-most part of the line 374 changes direction (herein referred to as “direction transition point DT”) to move from an upper position T1 as shown in FIG. 19 to a lower position T2 as shown in FIG. 20.

From above, it should be appreciated that movement of the actuator tube 322 by a first distance causes the distal end of the spring 320 to move by a second distance which is greater than the first distance. This occurs due the approximately 2-to-1 mechanical advantage achieved by the proximal portion 102, 104 of the catheter system 100 of FIGS. 18-32.

FIG. 20 shows the first proximal portion 102 of the catheter system 100 of FIGS. 18-32 connected to a fluid line of a dialysis machine such as line 16, 18 of the dialysis machine 10 (see also FIG. 2). In particular, a proximal part of the tube segment 101a, 101b has a coupling 342 that includes external threads. FIG. 20 shows an alternatively configured coupling 344 that may be used at the distal end of line 16, 18. The coupling 344 is configured to mate in a fluid tight manner to coupling 342. The coupling 344 includes a connector member having internal threads as shown in FIG. 20. The couplings 342, 344 are configured as Luer lock couplings which are well know in the medical device arts.

When the catheter system 100 of FIGS. 18-32 is desired to be connected to the line 16, 18 to perform a medical procedure, such as a dialysis procedure, the distal part of the line 16, 18 is urged against the proximal part of the actuator tube segment 322 until the couplings 342, 344 begin to mate with each other. Continued mating of the couplings 342, 344 results in a fluid tight connection between the catheter system 100 and the line 16, 18 as shown in FIG. 20. In this mated condition, the actuator tube 322 is now positioned in its lower position, and the proximal spring 318 is now in its compressed configuration. Also, the direction transition point DT of the line 374 is now in its lower position T2 as shown in FIG. 20.

When the catheter system 100 of FIGS. 18-32 is desired to be disconnected from the line 16, 18 after the medical procedure has been completed, the coupling 344 is manipulated so as to decouple or otherwise separate the couplings 342, 344 from each other. After decoupling, the distal part of the line 16, 18 is moved in a direction away from the proximal part of the tube segment 101a, 101b thereby allowing the actuator tube to 322 be urged by the proximal spring 318 back to its upper position shown in FIG. 19. Thereafter, a cap 346 that includes an elastomeric O-ring 347 is secured to the coupling 342 as shown in FIG. 19A in order to seal the catheter system 100 from outside contaminants. Note that the cap 346 is configured with an internal space 348 large enough so as to prevent actuation of the actuator tube 322. In other words, when the cap 348 is coupled to the proximal part of the tube segment 101a, 101b as shown in FIG. 19A, the proximal spring 318 is allowed to assume its expanded configuration thereby retaining the actuator tube 322 in its upper position. When the actuator tube 322 is retained in its upper position, the direction transition point DT of the line 374 is retained at its upper position T1 as shown in FIG. 19A. FIG. 18 shows the caps 346 of the proximal portions 102, 104 coupled to their respective proximal parts of the tube segments 101a, 101b. Also, each cap 346 may have an attachment assembly (not shown, but similar to attachment assembly 147 of FIG. 11) so that the cap 346 will not become lost or otherwise misplaced when decoupled from the tube segments 101a, 101b.

The distal portion 106 of the catheter system 100 of FIGS. 18-32 is shown in detail in FIGS. 21-22. The distal portion 106 is configured to possess a single component, i.e. the spring 320, that serves both the function of a spring and a conduit or cage. The spring 320 is secured to the distal part of the tube segment 101c as shown in FIGS. 21-22. The spring 320 possesses a distal opening 401, as well as numerous fluid openings 402 that extend through the walls of the spring 320.

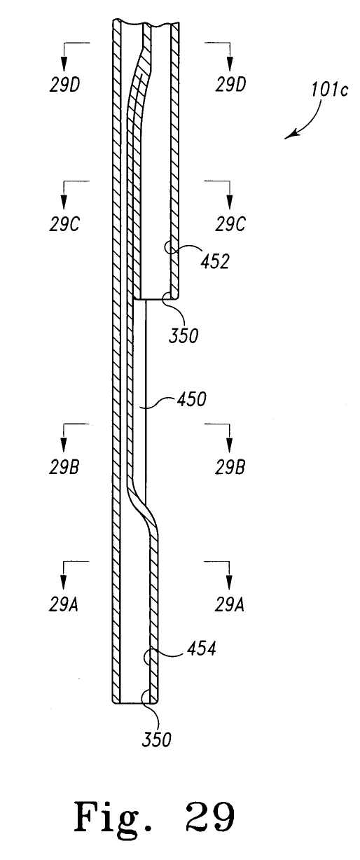

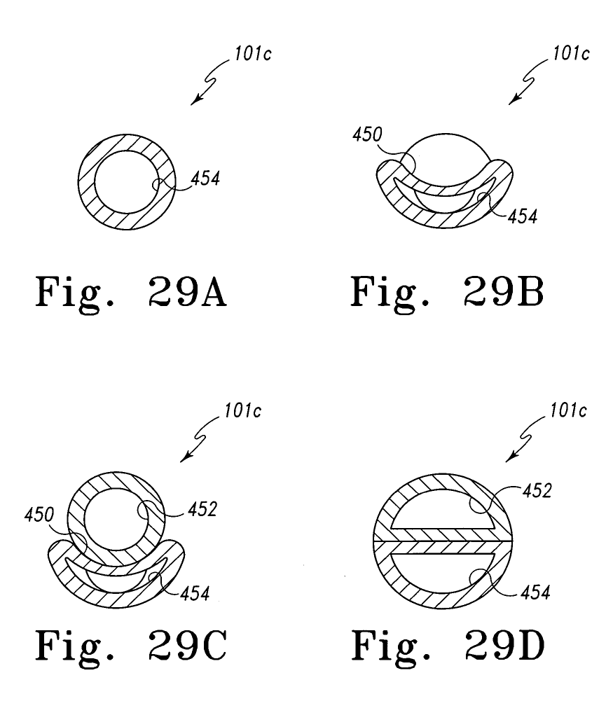

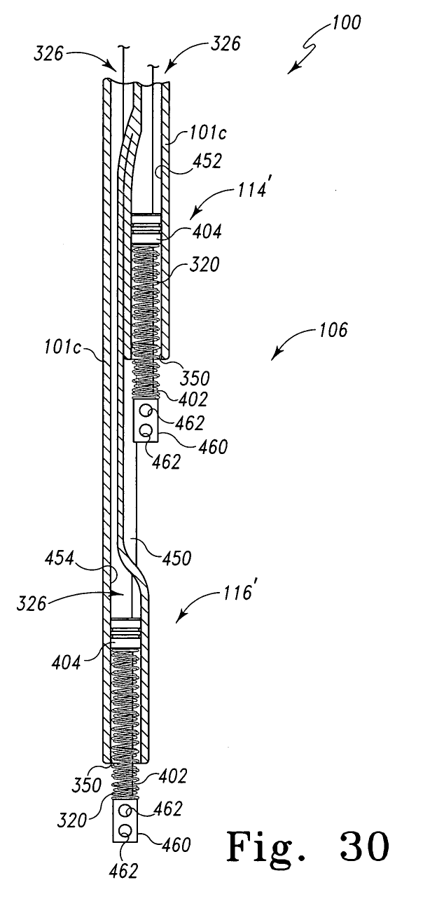

FIG. 29 shows the lower portion of the tube segment 101c with various components of the catheter system 100 removed for clarity of understanding, such as the distal spring 320, the bonding washer 404, and the linkage 326. The lower portion of the tube segment 101c is configured to define a recess 450 in which the proximal-most distal spring 320 is located as shown in FIGS. 21-22. The lower portion of the tube segment includes two lumens 452, 454 through which fluid, such as blood, may flow. (See, e.g., FIGS. 29 and 29A-29D.)

A proximal end of the spring 320 is secured to a bonding washer 404 as shown in FIGS. 21-22 and 26A-26B by, for example, welding. The washer 404, in turn, is secured to an internal sidewall of the tube segment 101c as shown in FIGS. 21-22. To this end, the washer 404 includes a plurality of annular grooves 406 that extend around the circumference of the washer. In order to secure the washer 404 to the tube segment 101c, the washer 404 is inserted into the passage defined by the tube segment to its position shown in FIGS. 21-22. Thereafter, heat is applied to the tube segment 101c around the outer periphery of the tube segment near the periphery of the washer 404. Such heat can be applied by a heated tube (i.e. a shrink tube) positioned at the outer periphery of the tube segment 101c. The heated tube applies an amount of heat sufficient to cause softening of the material of the tube segment (e.g. polyurethane) whereby softened material is driven into the grooves 406 of the washer 404. Pressure may be applied to the softened material to facilitate driving of the softened material into the grooves 406. Thereafter, cooling of the material of the tube segment 101c secures the washer 404 to the tube segment 101c.

As shown in FIGS. 21-22, the spring 320 is movable between a compressed configuration (FIG. 21) in which the spring 320 is completely contained within the distal part of the tube segment 101c, and an expanded configuration (FIG. 22) in which the spring 320 is partially positioned within the tube segment 101c but extends out through a distal orifice 350 thereof. In order to position the spring 320 in its compressed configuration as shown in FIG. 21, an upward force is applied to the cable 326 in the direction of arrow 352 in an amount sufficient to overcome the spring bias of spring 320 thereby compressing the spring and moving it completely within the distal part of the tube segment 101c as shown in FIG. 21.

In order to move the spring 320 to its expanded configuration as shown in FIG. 22, the force being applied to cable 326 in the direction of arrow 352 is removed thereby allowing the spring 320 to expand from its compressed configuration to its expanded configuration as shown in FIG. 22. Such expansion of the spring 320 causes the spring 320 to expand and thereby extend out of the distal orifice 350 of the tube segment 101c as shown in FIG. 22.

Note that in this embodiment of the distal portion 106, coupling of the line 16, 18 of the dialysis machine 10 to the catheter system 100 of FIGS. 18-32 causes the components of the first retractable conduit assembly 114′ to move from their positions shown in FIGS. 19 and 21 to their positions shown in FIGS. 20 and 22. Thus, the catheter system 100 of FIGS. 18-32 including the first retractable conduit assembly 114′ is shown in FIGS. 20 and 22 during a period of use such as during the performance of a dialysis procedure.

Also note that in this alternative embodiment, the springs 318 and 320 are configured such that spring 318 is the stronger one. In particular, during periods of non-use of the catheter system 100 of FIGS. 18-32, the bias of the proximal spring 318 overcomes the bias of the distal spring 320 so as to position the actuator tube 322 and the spring 320 in their upper positions as shown in FIGS. 19 and 21. However, when the influence of the spring 318 is removed from the spring 320 (such as when the catheter system 100 of FIGS. 18-32 is coupled to lines 16, 18), the spring 320 is configured to expand into its lower or expanded position as shown in FIG. 22 thereby extending out through the distal orifice 350 of the tube segment 101c.

It should be appreciated that constructing the linkage 326, 326′ to include the line 374 at its proximal portion is beneficial in the assembly of the catheter system 100. Indeed, the excess portion 410 (see FIGS. 19-20) of the line 374 on its proximal side is used to set the distal extent of the distal spring 320 in the tube segment 101c during assembly of the catheter system 100. To this end, the tube adapter 312 is configured with a channel 412 and an aperture 414 through which the line 374 may extend. Thus, during assembly, after the linkage 326, 326′ is positioned within the tube segments 101a, 101b, 101c and the bonding washer 404 is secured to the tube segment 101c, the distal end portion of the linkage 326, 326′ is then secured to the distal end of the spring 320. Thereafter, with the distal portion of the linkage 326, 326′ so secured, the proximal portion of the line 374 will extend within the channel 412 and out of the aperture 414 so as to position at least some of the excess portion 410 outside of the tube segments 101a, 101b. Thereafter, the proximal portion of the line 374 will be pulled so as to retract the spring 320 into the distal end portion of the tube assembly 101c until the distal end of the spring 320 is located at a desired position, such as the position shown in FIG. 21. Then, the proximal end of the line 374 will be attached to the tube segments 101a, 101b by adhesive such as a biocompatible cement or glue. After the line 374 is affixed to the tube segments 101a, 101b, the excess portion 410 of the line 374 may be clipped off so that no line 374 is located outside of the tube segments 101a, 101b as shown in FIG. 19A. It should be appreciated that the aperture 414 of the tube adapter 312 will be plugged up or otherwise sealed with cement or glue which is advanced and retained in the aperture 414 during the above line attachment process so that outside contaminants will not invade the catheter system 100.

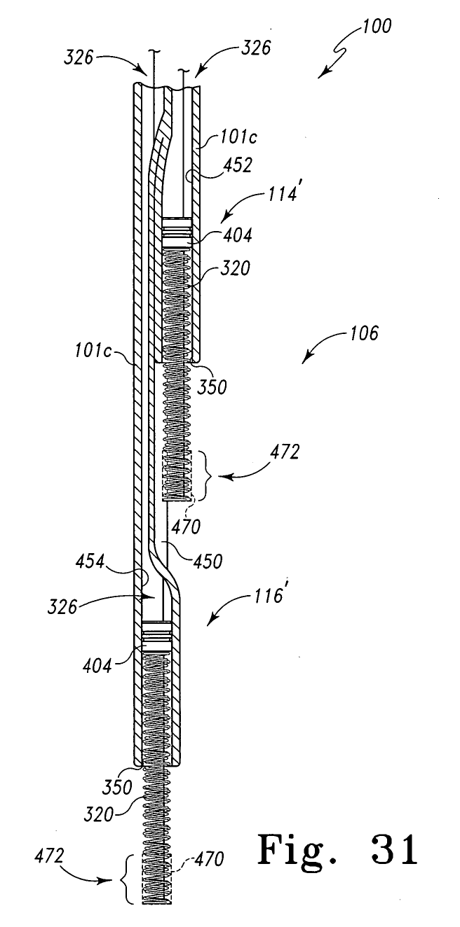

FIG. 30 shows an alternative embodiment of the distal portion 106 of the catheter system 100. In particular, an extension member 460 is attached to the distal end of each of the distal springs 320 as shown in FIG. 30. The extension member 460 is shaped as a cylinder and defines a central fluid passage. In addition, the extension member 460 has defined therein a plurality of side holes 462 as shown in FIG. 30.

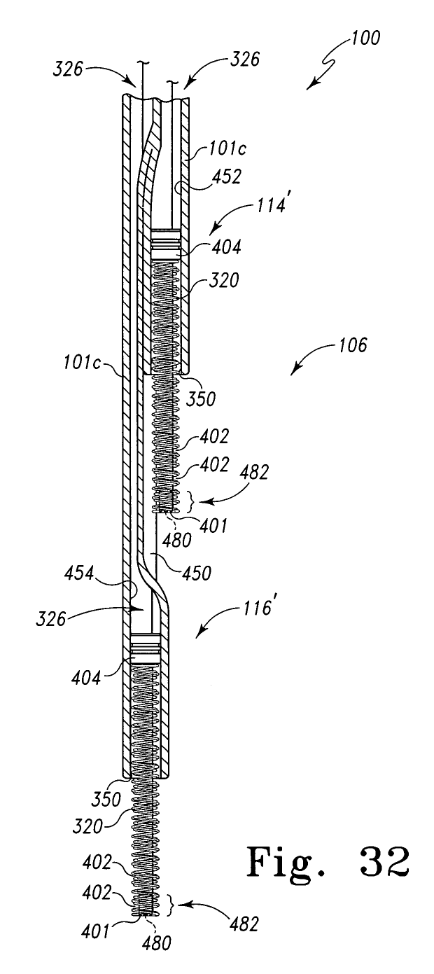

FIG. 31 shows another alternative embodiment of the distal portion 106 of the catheter system 100. In particular, a filler material 470 is secured to each of the distal springs within the interstices thereof. The filler material 470 may be a plastics material advanced into the interstices of the distal springs 320 in a molten state, and thereafter allowed to cool to form a cylindrical member. The filler material 470 may be located within the interstices of each of the distal springs at a lower portion 472 of the distal spring. The plastics material used as the filler material 470 may be a polyurethane, a polyethylene, or a polypropylene material, or may be any other plastics material that is capable of attaching itself to the distal springs when the material is melted and thereafter allowed to harden. It should be appreciated that the filler material, when hardened, forms a cylindrical member defining a central fluid passage.

FIG. 32 shows yet another alternative embodiment of the distal portion 106 of the catheter system 100. In particular, a plug 480 is positioned within the distal opening 401 of the distal spring 320. The plug 480 extends within the central fluid passage of the distal spring at a lower portion 482 thereof. In this embodiment, fluid would be prevented from flowing through the distal opening 401. However, fluid would still be able to flow into and out of the catheter system 100 through the interstices defined by the distal springs 320 (see, e.g., fluid openings 402).

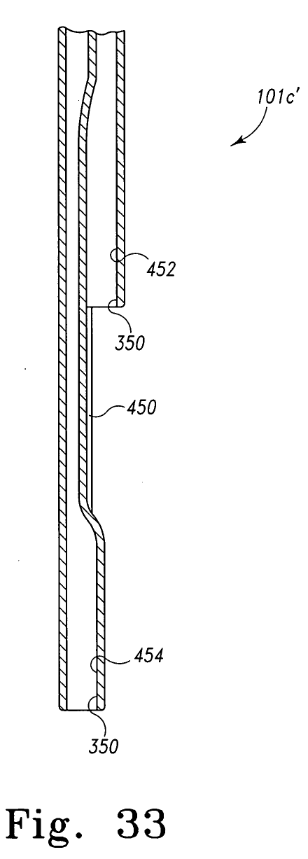

FIG. 33 shows an alternative embodiment of the tube segment of the distal portion of the catheter system of FIG. 18. In particular, FIG. 33 shows a tube segment 101c′ which may be substituted for the tube segment 101c of FIG. 29. In this embodiment, the narrowed part of the lumen 454 is enlarged (in relation to the embodiment of FIG. 29) by having only a single wall between the lower end portion of lumen 452 and the lumen 454 as shown in FIG. 33. Moreover, the single wall would continue distally until it widens so as to form the increased diameter portion of the distal end portion of the lumen 454 in which the distal spring 320 and the bonding washer 404 is housed.

There is a plurality of advantages arising from the various features of each of the embodiments of the catheter system 100 described herein. It will be noted that alternative embodiments the catheter system may not include all of the features described yet still benefit from at least some of the advantages of such features. Those of ordinary skill in the art may readily devise their own implementations of the catheter system that incorporate one or more of the features of the catheter system 100 and fall within the spirit and scope of the present invention as defined by the appended claims.

For example, any of the embodiments of the above-described dual-lumen catheter system 100 may be modified to incorporate any of the features of any of the catheter systems disclosed in any of U.S. Pat. Nos. 5,989,213; 6,156,016; 6,190,371; 6,475,207; 6,585,705; 6,723,084; 6,743,218; and 7,008,412, as well as, U.S. Patent Application Publication Nos. 2005/0096609 A1 and 2005/00559925 A1. The disclosures of each of the above-identified patents and published patent applications are hereby totally incorporated by reference in their entirety. For instance, any of the embodiments of the catheter system 100 may be modified to be a catheter system possessing only a single lumen such as disclosed in any of these identified U.S. patents and published U.S. patent application. Alternatively, any of the embodiments of the catheter system 100 may be modified to be a catheter system possessing more than two lumens, such as three lumens or four lumens. Moreover, the catheter system 100 could be modified to be a subcutaneous port catheter system that is implanted below the surface of a patient's skin as is described in the above-identified U.S. patents and published U.S. patent applications which are incorporated herein by reference.

It should be appreciated that the catheter system of the present disclosure may be used in its current state or with modifications thereto to perform any of the medical procedures disclosed in any of U.S. Pat. Nos. 5,989,213; 6,156,016; 6,190,371; 6,475,207; 6,585,705; 6,723,084; 6,743,218; and 7,008,412, as well as, U.S. Patent Application Publication Nos. 2005/0096609 A1 and 2005/00559925 A1.

For instance, while the above-described dual-lumen catheter system 100 was discussed as being effective to perform hemodialysis, the catheter system 100 can also be utilized to perform other medical procedures in which dual-lumen catheter access to the vascular system (e.g. the central venous system) is required. One example of such a medical procedure is plasmapheresis in which blood is withdrawn from the vascular system, components of the blood are separated outside of the body, and a portion of the blood components are then returned to the vascular system.

In addition, another medical procedure which may be performed using the above-described dual-lumen catheter system 100 is peritoneal dialysis. In particular, catheter system occlusion may be prevented during a peritoneal dialysis procedure in a manner similar to that described above with respect to the catheter system 100.

Moreover, if the catheter system 100 was modified to be a catheter system possessing only a single lumen, such modified catheter system could be used to perform medical procedures in which single-lumen catheter access to the vascular system is required. Examples of medical procedures in which single-lumen catheter access to the vascular system is required includes (i) chemotherapy or other long-term medicinal infusions, (ii) repetitive blood transfusions, (iii) repetitive blood samplings, and (iv) administration of total parenteral nutrition. Indeed, catheter system occlusion may be prevented during these medical procedures that utilize a single lumen catheter system in a manner similar to that described above with respect to the catheter system 100.

Furthermore, the above-described catheter system 100 was described as having a tissue ingrowth member (e.g. tissue ingrowth member 143) which is configured to facilitate attachment of the catheter system to the subcutaneous tissue 22 of the body. While the provision of such a tissue ingrowth member to effect attachment of such catheter system to the body of a patient has many advantages, the present catheter system may utilize other mechanisms which can function to attach the catheter system to the body on a long-term or even a short-term basis and still benefit from various advantages of other features of the present catheter system. An example of such an attachment mechanism is a plastic member having a hole or recess for receiving a catheter therein and further having one or more wing-like or flap-like extensions which may be sutured or taped to the skin of the patient 14. Additionally, it is possible that the above-described catheter system 100 may be modified so as to not include any mechanism which specifically functions to attach the catheter system to the body yet still benefits from some of the advantages of other features described herein.

While the catheter system 100 was described as being placed in the body 14 utilizing the permanent catheterization technique and has many advantages thereby, the catheter system 100 could be placed in the body 14 utilizing other techniques (e.g. a temporary catheterization technique) and still achieve some of the advantages of the catheter system 100 described herein.

Additionally, while the above-described catheter system 100 was described as being implanted in the body 14 so that a proximal portion of the catheter system is located external to the body 14 and the remainder of the catheter system is located within the body 14 (such as shown in FIG. 2), the catheter system 100 could be implanted entirely within the body and still achieve some of its advantages. More particularly, the catheter system 100 could be modified so as to be configured as a subcutaneous port catheter system having a retractable member (e.g. member 124, 200, 320) as shown and described herein. The subcutaneous port catheter system would be implanted entirely beneath the skin 20 of the body 14 within the subcutaneous tissue 22 (see FIGS. 15-17). Such subcutaneous port catheter system would be further configured and used as described in the above-identified U.S. patents and published U.S. patent applications, including U.S. Pat. No. 7,008,412, which are herein incorporated by reference.

What is claimed is:

1. A catheter system, comprising:
a structure defining a proximal orifice, a distal orifice, and a fluid passage extending therebetween;
an actuator movable in relation to said structure between a first location and a second location;
a member movable in relation to said structure between (i) a first position in which said member extends through said distal orifice so that a proximal part of said member is positioned within said fluid passage and a distal part of said member is positioned outside of said fluid passage, and (ii) a second position in which said member is entirely located within said fluid passage; and
a linkage connected between said actuator and said member,
wherein movement of said actuator from said second location to said first location causes movement of said member from said second position to said first position.

2. The catheter system of claim 1, wherein:
said member includes a first spring supported by said structure and configurable between an expanded configuration and a compressed configuration,
said first spring is in said expanded configuration when said member is in said first position, and
said first spring is in said compressed configuration when said member is in said second position.

3. The catheter system of claim 2, further comprising (i) a second spring supported by said structure and configured to urge said actuator from said first location toward said second location.

4. The catheter system of claim 3, wherein:
said first spring is configured to generate a first magnitude of spring force, and
said second spring is configured to generate a second magnitude of spring force which is greater than said first magnitude of spring force.

5. The catheter system of claim 4, wherein:
compression of said second spring causes said first spring to move from said compressed configuration to said expanded configuration, and
expansion of said second spring causes said first spring to move from said expanded configuration to said compressed configuration.

6. The catheter system of claim 3, wherein said second spring is positioned within said fluid passage.

7. The catheter system of claim 1, wherein said member includes a cage having a plurality of intersecting bars.

8. The catheter system of claim 7, further comprising a first spring positioned within said fluid passage and configured to urge said member from said second position towards said first position.

9. The catheter system of claim 8, wherein:
said first spring is configurable between a compressed configuration and an expanded configuration, and
movement of said first spring from said compressed configuration to said expanded configuration causes said member to move from said second position to said first position.

10. The catheter system of claim 9, further comprising (i) a second spring supported by said structure and configured to urge said actuator from said first location toward said second location.

11. The catheter system of claim 10, wherein:
said first spring is configured to generate a first magnitude of spring force, and
said second spring is configured to generate a second magnitude of spring force which is greater than said first magnitude of spring force.

12. The catheter system of claim 11, wherein:
compression of said second spring causes said first spring to move from said compressed configuration to said expanded configuration, and
expansion of said second spring causes said first spring to move from said expanded configuration to said compressed configuration.

13. The catheter system of claim 12, wherein both said first spring and said second spring are positioned within said fluid passage.

14. The catheter system of claim 1, wherein said structure includes a first coupling configured to connect to a second coupling of a fluid line of a dialysis machine.

15. The catheter system of claim 1, wherein:
said actuator defines a passageway,
a distal end of said linkage is connected to said member,
a proximal end of said linkage is connected to a proximal portion of said structure, and
said linkage extends through said passageway of said actuator.

16. The catheter system of claim 15, wherein:
movement of said actuator by a first distance D1 causes a distal end of said member to move by a second distance D2, and
D2 is greater than D1.

17. The catheter system of claim 1, wherein:
a distal end of said linkage is connected to said member, and
a proximal end of said linkage is connected to said actuator.

18. The catheter system of claim 17, wherein:
movement of said actuator by a first distance D1 causes a distal end of said member to move by a second distance D2, and
D2 is approximately equal to D1.

19. The catheter system of claim 1, wherein:
said linkage includes an inner line and an outer sheath positioned around said inner line, and
a distal end of said inner line is connected to said member.

20. The catheter system of claim 19, wherein:
said linkage further includes an auxiliary line,
a proximal end of said inner line is connected to a distal end of said auxiliary line, and
a proximal end of said auxiliary line is connected to a proximal portion of said structure,
said actuator defines a passageway, and
said auxiliary line extends through said passageway of said actuator.

21. The catheter system of claim 1, wherein said structure includes a first coupling positioned adjacent to said proximal orifice of said structure, further comprising:
a cap having a second coupling configured to cooperate with said first coupling to couple said cap to said structure, and
an attachment assembly including (i) a ring member positioned around said structure, and (ii) an arm attached between said cap and said ring member.

22. The catheter system of claim 1, wherein said member defines a proximal opening, a distal opening, and a central passage extending therebetween, further comprising:
a plug positioned in said central passage.

23. The catheter system of claim 1, wherein said member includes a spring that defines a plurality of interstices, further comprising:
a filler material positioned within said plurality of interstices at a distal portion of said spring.

24. A catheter system, comprising:
a structure defining a proximal orifice, a distal orifice, and a fluid passage;
an actuator movable in relation to said structure; and
a member movable in relation to said structure between (i) a first position in which said member is at least partially advanced outside of said structure through said distal orifice so that a distal end of said member is located at a first location outside of said fluid passage, and (ii) a second position in which said distal end of said member is located at a second location which is proximal to said first location,
wherein movement of said actuator causes movement of said member from said second position to said first position.

25. The catheter system of claim 24, wherein:
said member includes a first spring supported by said structure and configurable between an expanded configuration and a compressed configuration,
said first spring is in said expanded configuration when said member is in said first position, and
said first spring is in said compressed configuration when said member is in said second position.

26. The catheter system of claim 25, further comprising (i) a second spring supported by said structure and configured to urge said actuator in a proximal direction.

27. The catheter system of claim 26, wherein:
said first spring is configured to generate a first magnitude of spring force, and
said second spring is configured to generate a second magnitude of spring force which is greater than said first magnitude of spring force.

28. The catheter system of claim 27, wherein:
compression of said second spring causes said first spring to move from said compressed configuration to said expanded configuration, and
expansion of said second spring causes said first spring to move from said expanded configuration to said compressed configuration.

29. The catheter system of claim 26, wherein said second spring is positioned within said fluid passage.

30. The catheter system of claim 24, wherein said member includes a cage having a plurality of intersecting bars.

31. The catheter system of claim 30, further comprising a first spring positioned within said fluid passage and configured to urge said member from said second position towards said first position.

32. The catheter system of claim 31, wherein:
said first spring is configurable between a compressed configuration and an expanded configuration, and
movement of said first spring from said compressed configuration to said expanded configuration causes said member to move from said second position to said first position.

33. The catheter system of claim 32, further comprising (i) a second spring supported by said structure and configured to urge said actuator in a proximal direction.

34. The catheter system of claim 33, wherein:
said first spring is configured to generate a first magnitude of spring force, and
said second spring is configured to generate a second magnitude of spring force which is greater than said first magnitude of spring force.

35. The catheter system of claim 34, wherein:
compression of said second spring causes said first spring to move from said compressed configuration to said expanded configuration, and
expansion of said second spring causes said first spring to move from said expanded configuration to said compressed configuration.

36. The catheter system of claim 35, wherein both said first spring and said second spring are positioned within said fluid passage.

37. The catheter system of claim 24, wherein said structure includes a first coupling configured to connect to a second coupling of a fluid line of a dialysis machine.

38. The catheter system of claim 24, further comprising a linkage connected between said actuator and said member, wherein:
said actuator defines a passageway,
a distal end of said linkage is connected to said member,
a proximal end of said linkage is connected to a proximal portion of said structure, and
said linkage extends through said passageway of said actuator.

39. The catheter system of claim 38, wherein:
movement of said actuator by a first distance D1 causes a distal end of said member to move by a second distance D2, and
D2 is greater than D1.

40. The catheter system of claim 24, further comprising a linkage connected between said actuator and said member wherein:
a distal end of said linkage is connected to said member, and
a proximal end of said linkage is connected to said actuator.

41. The catheter system of claim 40, wherein:
movement of said actuator by a first distance D1 causes a distal end of said member to move by a second distance D2, and
D2 is approximately equal to D1.

42. The catheter system of claim 24, further comprising a linkage connected between said actuator and said member wherein:
said linkage includes an inner line and an outer sheath positioned around said inner line, and
a distal end of said inner line is connected to said member.

43. The catheter system of claim 42, wherein:
said linkage further includes an auxiliary line,
a proximal end of said inner line is connected to a distal end of said auxiliary line, and
a proximal end of said auxiliary line is connected to a proximal portion of said structure,
said actuator defines a passageway, and
said auxiliary line extends through said passageway of said actuator.

44. The catheter system of claim 24, wherein said structure includes a first coupling positioned adjacent to said proximal orifice of said structure, further comprising:
a cap having a second coupling configured to cooperate with said first coupling to couple said cap to said structure, and
an attachment assembly including (i) a ring member positioned around said structure, and (ii) an arm attached between said cap and said ring member.

45. The catheter system of claim 24, wherein said member defines a proximal opening, a distal opening, and a central passage extending therebetween, further comprising:
a plug positioned in said central passage.

46. The catheter system of claim 24, wherein said member includes a spring that defines a plurality of interstices, further comprising:
a filler material positioned within said plurality of interstices at a distal portion of said spring.

47. A catheter system, comprising:
a structure defining a proximal orifice, a distal orifice, and a fluid passage;
an actuator movable in relation to said structure;
a member movable in relation to said structure between (i) a first position in which said member is at least partially advanced outside of said structure through said distal orifice so that a distal end of said member is located outside of said fluid passage, and (ii) a second position in which said distal end of said member is located within said fluid passage; and
a linkage connected between said actuator and said member,
wherein movement of said actuator causes movement of said member from said second position to said first position.

48. The catheter system of claim 47, wherein:
said member includes a first spring supported by said structure and configurable between an expanded configuration and a compressed configuration,
said first spring is in said expanded configuration when said member is in said first position, and
said first spring is in said compressed configuration when said member is in said second position.

49. The catheter system of claim 48, further comprising (i) a second spring supported by said structure and configured to urge said actuator in a proximal direction.

50. The catheter system of claim 49, wherein:
said first spring is configured to generate a first magnitude of spring force, and
said second spring is configured to generate a second magnitude of spring force which is greater than said first magnitude of spring force.

51. The catheter system of claim 50, wherein:
compression of said second spring causes said first spring to move from said compressed configuration to said expanded configuration, and
expansion of said second spring causes said first spring to move from said expanded configuration to said compressed configuration.

52. The catheter system of claim 49, wherein said second spring is positioned within said fluid passage.

53. The catheter system of claim 47, wherein said member includes a cage having a plurality of intersecting bars.

54. The catheter system of claim 53, further comprising a first spring positioned within said fluid passage and configured to urge said member from said second position towards said first position.

55. The catheter system of claim 54, wherein:
said first spring is configurable between a compressed configuration and an expanded configuration, and
movement of said first spring from said compressed configuration to said expanded configuration causes said member to move from said second position to said first position.

56. The catheter system of claim 55, further comprising (i) a second spring supported by said structure and configured to urge said actuator in a proximal direction.

57. The catheter system of claim 56, wherein:
said first spring is configured to generate a first magnitude of spring force, and
said second spring is configured to generate a second magnitude of spring force which is greater than said first magnitude of spring force.

58. The catheter system of claim 57, wherein:
compression of said second spring causes said first spring to move from said compressed configuration to said expanded configuration, and
expansion of said second spring causes said first spring to move from said expanded configuration to said compressed configuration.

59. The catheter system of claim 58, wherein both said first spring and said second spring are positioned within said fluid passage.

60. The catheter system of claim 47, wherein said structure includes a first coupling configured to connect to a second coupling of a fluid line of a dialysis machine.

61. The catheter system of claim 47, wherein:
said actuator defines a passageway,
a distal end of said linkage is connected to said member,
a proximal end of said linkage is connected to a proximal portion of said structure, and
said linkage extends through said passageway of said actuator.

62. The catheter system of claim 61, wherein:
movement of said actuator by a first distance D1 causes a distal end of said member to move by a second distance D2, and
D2 is greater than D1.

63. The catheter system of claim 47, wherein:
a distal end of said linkage is connected to said member, and
a proximal end of said linkage is connected to said actuator.

64. The catheter system of claim 63, wherein:
movement of said actuator by a first distance D1 causes a distal end of said member to move by a second distance D2, and
D2 is approximately equal to D1.

65. The catheter system of claim 47, wherein:
said linkage includes an inner cable and an outer sheath positioned around said inner cable, and
a distal end of said inner cable is connected to said member.

66. The catheter system of claim 65, wherein:
said linkage further includes an auxiliary line,
a proximal end of said inner cable is connected to a distal end of said auxiliary line, and
a proximal end of said auxiliary line is connected to a proximal portion of said structure,
said actuator defines a passageway, and
said auxiliary line extends through said passageway of said actuator.

67. The catheter system of claim 47, wherein said structure includes a first coupling positioned adjacent to said proximal orifice of said structure, further comprising:
a cap having a second coupling configured to cooperate with said first coupling to couple said cap to said structure, and
an attachment assembly including (i) a ring member positioned around said structure, and (ii) an arm attached between said cap and said ring member.

68. The catheter system of claim 47, wherein said member defines a proximal opening, a distal opening, and a central passage extending therebetween, further comprising:
a plug positioned in said central passage.

69. The catheter system of claim 47, wherein said member includes a spring that defines a plurality of interstices, further comprising:
a filler material positioned within said plurality of interstices at a distal portion of said spring.

70. The catheter system of claim 19, wherein:
said inner line includes a nylon monofilament line, and
said outer sheath is positioned around said nylon monofilament line.

71. The catheter system of claim 42, wherein:
said inner line includes a nylon monofilament line, and
said outer sheath is positioned around said nylon monofilament line.

72. The catheter system of claim 1, wherein:
said structure includes a proximal end portion that defines said proximal orifice,
said proximal end portion is configured to receive therein a male portion of a Luer lock coupling, said male portion having a fluid channel defined therethrough, and
advancement of said male portion of said Luer lock coupling into said proximal end portion through said proximal orifice causes movement of said actuator from said second location to said first location.

73. The catheter system of claim 24, wherein:
said structure includes a proximal end portion that defines said proximal orifice,
said proximal end portion is configured to receive therein a male portion of a Luer lock coupling, and
advancement of said male portion of said Luer lock coupling into said proximal end portion through said proximal orifice causes movement of said actuator.

74. The catheter system of claim 47, wherein:
said structure includes a proximal end portion that defines said proximal orifice,
said proximal end portion is configured to receive therein a male portion of a Luer lock coupling, and
advancement of said male portion of said Luer lock coupling into said proximal end portion through said proximal orifice causes movement of said actuator.
